(19) United States
(12) Patent Application Publication
(10) Pub. No.: US 2023/0181016 A1
(43) Pub. Date: Jun. 15, 2023

(54) IMAGING SYSTEM

(71) Applicant: Gentuity, LLC, Sudbury, MA (US)

(72) Inventors: Giovanni J. Ughi, Arlington, MA (US); Hendrick Melo, Lexington, MA (US); Taylor Braun-Jones, Brookline, MA (US); Christopher A. Battles, Seymour, CT (US); Christopher Petroff, Groton, MA (US); Christopher Petersen, Carlisle, MA (US); Keith Boudreau, North Andover, MA (US); R. Maxwell Flaherty, Topsfield, MA (US); J. Christopher Flaherty, Nottingham, NH (US)

(21) Appl. No.: 17/919,809

(22) PCT Filed: Apr. 29, 2021
(86) PCT No.: PCT/US21/29836

§ 371 Date: Oct. 19, 2022

Related U.S. Application Data
(60) Provisional application No. 63/017,258, filed on Apr. 29, 2020.
(60) Provisional application No. 63/154,934, filed on Mar. 1, 2021.

(51) Int. Cl. A61B 1/04; A61B 1/07

(52) U.S. Cl. CPC A61B 1/04; A61B 1/07

(57) ABSTRACT

Provided herein are imaging systems for a patient comprising an imaging probe and an imaging assembly. The imaging probe comprises: an elongate shaft comprising a proximal end, a distal portion, and a lumen extending between the proximal end and the distal portion; a rotatable optical core comprising a proximal end and a distal end, wherein at least a portion of the rotatable optical core is positioned within the lumen of the elongate shaft; and an optical assembly positioned proximate the distal end of the rotatable optical core, the optical assembly configured to direct light to tissue to be imaged and collect reflected light from the tissue to be imaged. The imaging assembly is constructed and arranged to optically couple to the imaging probe. The imaging assembly is configured to emit light into the imaging probe and receive the reflected light collected by the optical assembly.

### RELATED APPLICATIONS

This application claims benefit of U.S. Provisional Application Ser. No. 63/017,258, titled “Imaging System”, filed Apr. 29, 2020, the content of which is incorporated by reference in its entirety.

This application claims benefit of U.S. Provisional Application Ser. No. 63/154,934, titled “Optical Imaging Systems”, filed Mar. 1, 2021, the content of which is incorporated by reference in its entirety.

This application is related to U.S. Provisional Application Ser. No. 62/148,355, titled “Micro-Optic Probes for Neurology”, filed Apr. 16, 2015, the content of which is incorporated by reference in its entirety.

This application is related to U.S. Provisional Application Ser. No. 62/322,182, titled “Micro-Optic Probes for Neurology”, filed Apr. 13, 2016, the content of which is incorporated by reference in its entirety.

This application is related to International PCT Patent Application Serial Number PCT/US2016/027764, titled “Micro-Optic Probes for Neurology” filed Apr. 15, 2016, Publication Number WO 2016/168605, published Oct. 20, 2016, the content of which is incorporated by reference in its entirety.

This application is related to U.S. patent application Ser. No. 15/566,041, titled “Micro-Optic Probes for Neurology”, filed Oct. 12, 2017, United States Publication Number 2018-0125372, published May 10, 2018, the content of which is incorporated by reference in its entirety.

This application is related to U.S. Provisional Application Ser. No. 62/212,173, titled “Imaging System Includes Imaging Probe and Delivery Devices”, filed Aug. 31, 2015, the content of which is incorporated by reference in its entirety.

This application is related to U.S. Provisional Application Ser. No. 62/368,387, titled “Imaging System Includes Imaging Probe and Delivery Devices”, filed Jul. 29, 2016, the content of which is incorporated by reference in its entirety.

This application is related to International PCT Patent Application Serial Number PCT/US2016/049415, titled “Imaging System Includes Imaging Probe and Delivery Devices”, filed Aug. 30, 2016, Publication Number WO 2017/040484, published Mar. 9, 2017, the content of which is incorporated by reference in its entirety.

This application is related to U.S. patent application Ser. No. 15/751,570, titled “Imaging System Includes Imaging Probe and Delivery Devices”, filed Feb. 9, 2018, U.S. Pat. No. 10,631,718, issued Apr. 28, 2020, the content of which is incorporated by reference in its entirety.

This application is related to U.S. patent application Ser. No. 16/820,991, titled “Imaging System Includes Imaging Probe and Delivery Devices”, filed Mar. 17, 2020, Publication Number 2021-0045622, published Feb. 18, 2021, the content of which is incorporated by reference in its entirety.

This application is related to U.S. Provisional Application Ser. No. 62/591,403, titled “Imaging System”, filed Nov. 28, 2017, the content of which is incorporated by reference in its entirety.

This application is related to U.S. Provisional Application Ser. No. 62/671,142, titled “Imaging System”, filed May 14, 2018, the content of which is incorporated by reference in its entirety.

This application is related to International PCT Patent Application Serial Number PCT/US2018/062766, titled “Imaging System”, filed Nov. 28, 2018, Publication Number WO 2019/108598, published Jun. 6, 2019, the content of which is incorporated by reference in its entirety.

This application is related to U.S. patent application Ser. No. 16/764,087, titled “Imaging System”, filed May 14, 2020, Publication Number 2020-0288950, published Sep. 17, 2020, the content of which is incorporated by reference in its entirety.

This application is related to U.S. Provisional Application Ser. No. 62/732,114, titled “Imaging System with Optical Pathway”, filed Sep. 17, 2018, the content of which is incorporated by reference in its entirety.

This application is related to International PCT Patent Application Serial Number PCT/US2019/051447, titled “Imaging System with Optical Pathway”, filed Sep. 17, 2019, Publication Number WO 2020/061001, published Mar. 26, 2020, the content of which is incorporated by reference in its entirety.

This application is related to U.S. patent application Ser. No. 17/276,500, filed Mar. 16, 2021, titled “Imaging system with Optical Pathway”, Publication Number ______, published ______, the content of which is incorporated by reference in its entirety.

This application is related to U.S. Provisional Application Ser. No. 62/840,450, titled “Imaging Probe with Fluid Pressurization Element”, filed Apr. 30, 2019, the content of which is incorporated by reference in its entirety.

This application is related to International PCT Patent Application Serial Number PCT/US2020/030616, titled “Imaging Probe with Fluid Pressurization Element”, filed Apr. 30, 2020, Publication Number WO 2020/223433, published Nov. 5, 2020, the content of which is incorporated by reference in its entirety.

This application is related to U.S. Provisional Application Ser. No. 62/850,945, titled “OCT-Guided Treatment of a Patient”, filed May 21, 2019, the content of which is incorporated by reference in its entirety.

This application is related to U.S. Provisional Application Ser. No. 62/906,353, titled “OCT-Guided Treatment of a Patient”, filed Sep. 26, 2019, the content of which is incorporated by reference in its entirety.

This application is related to International PCT Patent Application Serial Number PCT/US2020/033953, titled “Systems and Methods for OCT-Guided Treatment of a Patient”, filed May 21, 2020, Publication Number WO 2020/237024, published Nov. 26, 2020, the content of which is incorporated by reference in its entirety.

### FIELD OF THE INVENTION

The present invention relates generally to imaging systems, and in particular, intravascular imaging systems including imaging probes and delivery devices.

### BACKGROUND

Imaging probes have been commercialized for imaging various internal locations of a patient, such as an intravascular probe for imaging a patient's heart. Current imaging probes are limited in their ability to reach certain anatomical locations due to their size and rigidity. Current imaging probes are inserted over a guidewire, which can compromise their placement and limit use of one or more delivery catheters through which the imaging probe is inserted. There is a need for imaging systems that include probes with reduced diameter and high flexibility, as well as systems with one or more delivery devices compatible with these improved imaging probes.

### SUMMARY

According to an aspect of the present inventive concepts, an imaging system for a patient comprises an imaging probe, an optical assembly, and an imaging assembly. The imaging probe comprises: an elongate shaft comprising a proximal end, a distal portion, and a lumen extending between the proximal end and the distal portion; a rotatable optical core comprising a proximal end and a distal end, and at least a portion of the rotatable optical core is positioned within the lumen of the elongate shaft. The optical assembly is positioned proximate the distal end of the rotatable optical core and is configured to direct light to tissue to be imaged and collect reflected light from the tissue to be imaged. The imaging assembly is constructed and arranged to optically couple to the imaging probe and is configured to emit light into the imaging probe and receive the reflected light collected by the optical assembly.

In some embodiments, the system further comprises a retraction assembly comprising a carrier, and the imaging probe comprises a pullback connector, and the carrier is configured to removably attach to the pullback connector. The system further comprises a delivery catheter operably attached to the retraction assembly and through which the imaging probe can be inserted, and the carrier can comprise a two-piece assembly configured to enable operator adjustment of the position of the pullback connector relative to the delivery catheter. The two-piece assembly can be configured to adjust the position of the pullback connector relative to the delivery catheter up to a distance of at least 5 mm, 7 mm, and/or 15 mm. The two-piece assembly can be configured to adjust the position of the pullback connector relative to the delivery catheter in increments as small as 1.0 mm, 0.7 mm, 0.5 mm, and/or 0.3 mm.

In some embodiments, the system can further comprise an algorithm configured to determine persist time. The algorithm can be further configured to calculate a vascular flow dynamic parameter based on the determined persist time.

In some embodiments, the system is configured to perform a pullback procedure, and the system is further configured to initiate the pullback procedure based on detection of a T-wave.

In some embodiments, the system is configured to perform a pullback procedure, and the system is further configured to initiate the pullback procedure based on an analysis of an angiographic image.

In some embodiments, the system is configured to perform a pullback procedure, and the system is further configured to initiate the pullback procedure based on an analysis of an EKG signal (e.g. an EKG signal produced via an analysis of OCT data by an algorithm of the system).

In some embodiments, the system is configured to perform a pullback procedure, and the system is further configured to initiate the pullback procedure based on detection of clearing of blood from a location to be imaged.

In some embodiments, the system is configured to perform a pullback procedure, and the system is further configured to initiate the pullback procedure based on presence of two triggering conditions at the same time. A first triggering condition can comprise detection of clearing of blood from a location to be imaged, and the second triggering condition can be based on an analysis of an EKG signal.

In some embodiments, the optical assembly is configured to be positioned in a first vessel, and the system is configured to produce an image of a target location outside of the first vessel. The first vessel can comprise a vessel of the brain, and the target location can comprise a location in the brain outside of the vessel of the brain. The light can be directed to tissue and/or the light collected from tissue passes through cerebral spinal fluid. The target location can comprise locations including one or more physiologic markers of disease, such as: tumor tissue, neuritic plaques, amyloid plaques, cerebral infarcts, atherosclerosis, and/or other tissue associated with a disease or disorder of the patient. The target location can comprise locations within a blood vessel, locations outside of a blood vessel, perivascular structures, subarachnoid space, and/or arachnoid trabeculae.

In some embodiments, the system is configured to display image data in a first mode comprising an oblique representation of the imaged tissue, and in a second mode comprising a fly-through representation of the imaged tissue. The system can be configured to allow an operator to transition between the first mode and the second mode.

In some embodiments, the optical assembly is configured to be positioned within a lumen of the intracranial vasculature to produce an image of one or more physiologic markers of vascular dementia and/or Alzheimer's disease (e.g. where these physiologic markers are at an anatomic location within the lumen and/or outside of the lumen in which the optical assembly is positioned). The physiologic markers can comprise one, two, or more markers selected from the group consisting of: amyloid plaques; neuritic plaques; cerebral infarcts; atherosclerosis; and combinations thereof.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of an imaging system comprising an imaging probe and independent retraction and rotation assemblies, consistent with the present inventive concepts.

FIG. 1A illustrates a schematic view of an imaging system comprising an imaging probe operably attachable to a patient interface module, and an independent pullback module operably attachable to the patient interface module and the imaging probe, consistent with the present inventive concepts.

FIG. 1B illustrates a schematic view of an imaging system comprising an imaging probe operably attachable to a module comprising a first connector for attaching to a rotation motive element and a second connector for attaching to a retraction motive element, consistent with the present inventive concepts.

FIG. 2A illustrates a perspective view of connectors being attached to a patient interface module, consistent with the present inventive concepts.

FIG. 2B illustrates a perspective view of a pullback housing, consistent with the present inventive concepts.

FIG. 3 illustrates a perspective view of connectors being attached to a patient interface module, consistent with the present inventive concepts.

FIG. 4 illustrates a perspective view of an adaptor for use with an imaging system, consistent with the present inventive concepts.

FIGS. 5A and 5B illustrate a perspective view of a pullback module operably attached to a portion of an imaging probe and a delivery catheter, and an exploded view of a portion of a pullback module, respectively, consistent with the present inventive concepts.

FIG. 6 illustrates a flow chart of a method of calculating a fractional flow reserve (FFR) using OCT, consistent with the present inventive concepts.

FIG. 7 illustrates a flow chart of a method of triggering a pullback imaging procedure, consistent with the present inventive concepts.

FIG. 8 illustrates a flow chart of a method of triggering a pullback imaging procedure based on multiple triggers, consistent with the present inventive concepts.

FIG. 9 illustrates an OCT image showing a vessel suspended in cerebral fluid, consistent with the present inventive concepts.

FIGS. 10A-10C illustrate various views of portions of an imaging probe, consistent with the present inventive concepts.

FIGS. 11A-11E illustrate five displays of lumen imaging data, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the terms “about” or “approximately” shall refer to ±30%.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below room pressure. As used herein, the term “vacuum” can be used to refer to a full or partial vacuum, or any negative pressure as described herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “lesion” comprises a segment of a blood vessel (e.g. an artery) that is in an undesired state. As used herein, lesion shall include a narrowing of a blood vessel (e.g. a stenosis), and/or a segment of a blood vessel, with or without narrowing, that includes a buildup of calcium, lipids, cholesterol, and/or other plaque.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are imaging systems for a patient comprising an imaging probe and an imaging assembly. The imaging probe comprises an elongate shaft, a rotatable optical core, and an optical assembly. The shaft comprises a proximal end, a distal portion, and a lumen extending between the proximal end and the distal portion. The rotatable optical core comprises a proximal end and a distal end, and at least a portion of the rotatable optical core is positioned within the lumen of the elongate shaft. The optical assembly is positioned proximate the distal end of the rotatable optical core, and it is configured to direct light to tissue and collect reflected light from the tissue. The imaging systems can comprise one or more algorithms configured to enhance the performance of the system.

The imaging systems of the present inventive concepts can be used to provide image data representing arteries, veins, and/or other body conduits, and to image one or more devices inserted into those conduits. The imaging system can be used to image tissue and/or other structures outside of the blood vessel and/or other lumen into which the imaging probe is inserted. The imaging systems can provide image data related to healthy tissue, as well a diseased tissue, such as blood vessels including a stenosis, myocardial bridge, and/or other vessel narrowing (“lesion” or “stenosis” herein), and/or blood vessels including an aneurysm. The systems can be configured to provide treatment information, such as when the treatment information is used by an operator (e.g. a clinician of the patient) to plan a treatment and/or to predict a treatment outcome.

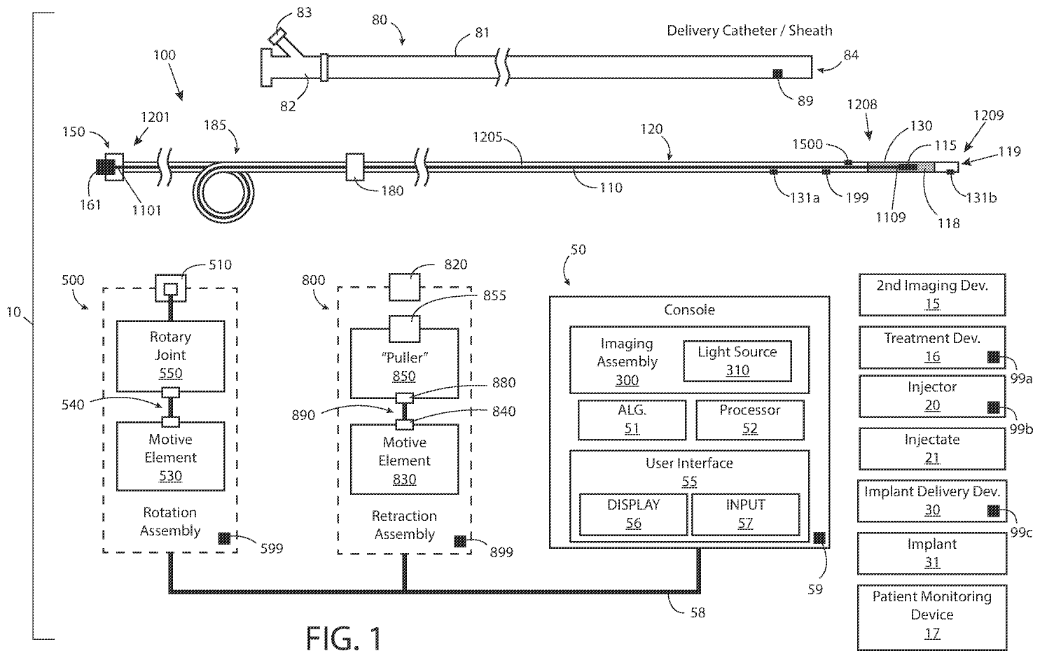

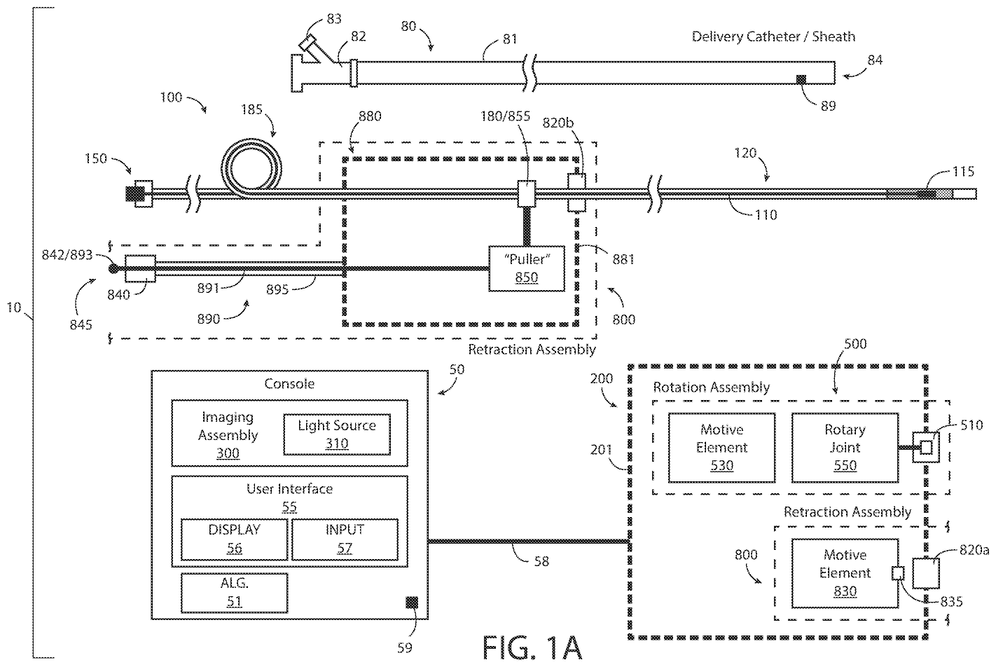

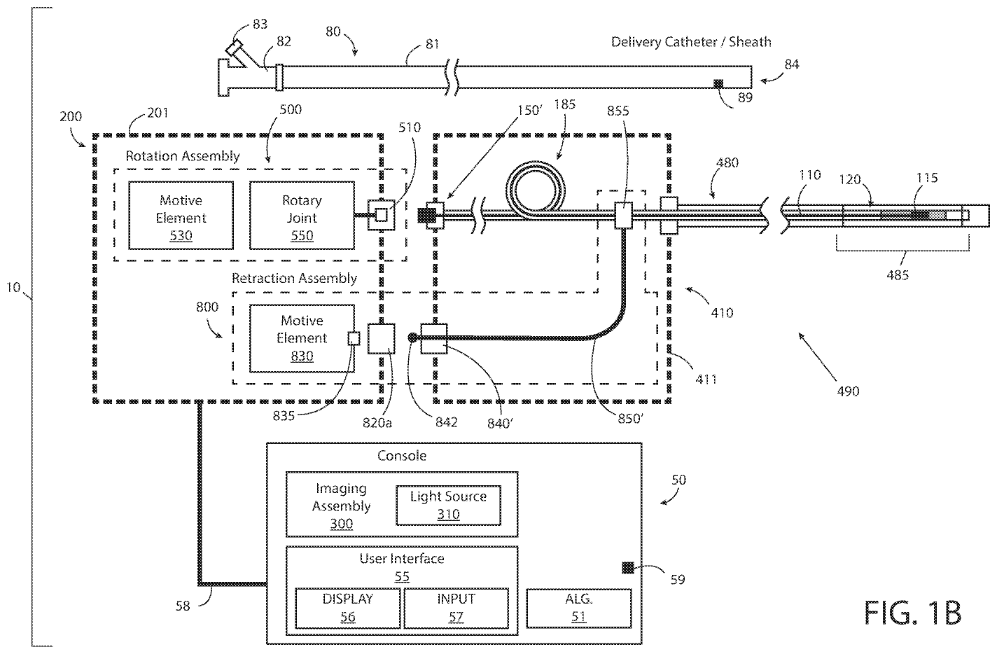

Referring now to FIG. 1, a schematic view of an imaging system comprising an imaging probe and independent retraction and rotation assemblies is illustrated, consistent with the present inventive concepts. Imaging system 10 is constructed and arranged to collect image data and produce one or more images based on the recorded data, such as when imaging system 10 comprises an Optical Coherence Tomography (OCT) imaging system constructed and arranged to collect image data (“image data” or “OCT data” herein) of an imaging location (e.g. a segment of a blood vessel or other vessel of the patient, such as during a pullback procedure). Imaging system 10 comprises catheter-based probe, imaging probe 100, as well as a rotation assembly 500 and a retraction assembly 800, each of which can operably attach to imaging probe 100. Imaging system 10 can further comprise console 50 which is configured to operably connect to imaging probe 100, such as via rotation assembly 500 and/or retraction assembly 800. Imaging probe 100 can be introduced into a conduit of the patient, such as a blood vessel or other conduit of the patient, using one or more delivery catheters, for example delivery catheter 80 shown. Additionally or alternatively, imaging probe 100 can be introduced through an introducer device, such as an endoscope, arthroscope, balloon dilator, or the like. In some embodiments, imaging probe 100 is configured to be introduced into a vessel of the patient selected from the group consisting of: an artery; a vein; an artery within or proximate the heart; a vein within or proximate the heart; an artery within or proximate the brain; a vein within or proximate the brain; a peripheral artery; a peripheral vein; a chamber of the patient; a channel of the patient; a canal of the patient (e.g. epidural space and/or intrathecal space of the patient's spine); a duct of the patient; a conduit or other internal location that is accessed through a natural body orifice, such as the esophagus; a body cavity or other internal location accessed through a surgically created orifice, such as the abdomen; and combinations of one or more of these. Imaging system 10 can further comprise multiple imaging devices, second imaging device 15 shown. Imaging system 10 can further comprise a device configured to treat the patient, treatment device 16. Imaging system 10 can further comprise one or more devices that are configured to monitor one, two, or more physiologic and/or other parameters of the patient, such as patient monitoring device 17 shown. Imaging system 10 can further comprise a fluid injector, such as injector 20, which can be configured to inject one or more fluids, such as a flushing fluid, an imaging contrast agent (e.g. a radiopaque contrast agent, hereinafter “contrast”) and/or other fluid, such as injectate 21 shown. Imaging system 10 can further comprise an implant, such as implant 31, which can be implanted in the patient via a delivery device, such as an implant delivery device 30 and/or delivery catheter 80.

In some embodiments, imaging probe 100 and/or another component of imaging system 10 can be of similar construction and arrangement to the similar components described in applicant's co-pending U.S. patent application Ser. No. 15/566,041, titled “Micro-Optic Probes for Neurology”, filed Oct. 12, 2017; the content of which is incorporated herein by reference in its entirety for all purposes. Imaging probe 100 can be constructed and arranged to collect image data from a patient site, such as an intravascular cardiac site, an intracranial site, or other site accessible via the vasculature of the patient. In some embodiments, imaging system 10 can be of similar construction and arrangement to the similar systems and their methods of use described in applicant's co-pending U.S. patent application Ser. No. 16/820,991, titled “Imaging System Includes Imaging Probe and Delivery Devices”, filed Mar. 27, 2020; the content of which is incorporated herein by reference in its entirety for all purposes.

Delivery catheter 80 comprises an elongate shaft, shaft 81, with a lumen 84 therethrough, and a connector 82 positioned on its proximal end. Connector 82 can comprise a Touhy or valved connector, such as a valved connector configured to prevent fluid egress from the associated delivery catheter 80 (with and/or without a separate shaft positioned within the connector 82). Connector 82 can comprise a port 83, such as a port constructed and arranged to allow introduction of fluid into delivery catheter 80 and/or for removing fluids from delivery catheter 80. In some embodiments, a flushing fluid, as described herein, is introduced via one or more ports 83, such as to remove blood or other undesired material from locations proximate optical assembly 115 (e.g. from a location proximal to optical assembly 115 to a location distal to optical assembly 115). Port 83 can be positioned on a side of connector 82 and can include a luer fitting and a cap and/or valve. Shafts 81, connectors 82, and ports 83 can each comprise standard materials and be of similar construction to commercially available introducers, guide catheters, diagnostic catheters, intermediate catheters and microcatheters used in interventional procedures. Delivery catheter 80 can comprise a catheter configured to deliver imaging probe 100 to an intracerebral location, an intracardiac location, and/or another location within a patient.

Imaging system 10 can comprise two or more delivery catheters 80, such as three or more delivery catheters 80. Multiple delivery catheters 80 can comprise at least a vascular introducer, and other delivery catheters 80 that can be inserted into the patient therethrough, after the vascular introducer is positioned through the skin of the patient. Two or more delivery catheters 80 can collectively comprise sets of inner diameters (IDs) and outer diameters (ODs) such that a first delivery catheter 80 slidingly receives a second delivery catheter 80 (e.g. the second delivery catheter OD is less than or equal to the first delivery catheter ID), and the second delivery catheter 80 slidingly receives a third delivery catheter 80 (e.g. the third delivery catheter OD is less than or equal to the second delivery catheter ID), and so on. In these configurations, the first delivery catheter 80 can be advanced to a first anatomical location, the second delivery catheter 80 can be advanced through the first delivery catheter to a second anatomical location distal or otherwise remote (hereinafter “distal”) to the first anatomical location, and so on as appropriate, using sequentially smaller diameter delivery catheters 80. In some embodiments, delivery catheters 80 can be of similar construction and arrangement to the similar components described in applicant's co-pending U.S. patent application Ser. No. 16/820,991, titled “Imaging System Includes Imaging Probe and Delivery Devices”, filed Mar. 27, 2020; the content of which is incorporated herein by reference in its entirety for all purposes.

Imaging probe 100 comprises an elongate body, comprising one or more elongate shafts and/or tubes, elongate shaft 120 herein. Shaft 120 comprises a proximal end 1201, distal end 1209, and a lumen 1205 extending therebetween. In some embodiments, lumen 1205 can include multiple coaxial lumens within the one or more elongate shafts 120, such as one or more lumens abutting each other to define a single lumen 1205. In some embodiments, at least a portion of shaft 120 comprises a torque shaft. In some embodiments, a portion of shaft 120 comprises a braided construction. In some embodiments, a portion of shaft 120 comprises a spiral cut tube (e.g. a spiral cut metal tube). In some embodiments, the pitch of the spiral cut can be varied along the length of the cut, such as to vary the stiffness of shaft 120 along the cut. A portion of shaft 120 can comprise a tube constructed of nickel-titanium alloy. Shaft 120 operably surrounds a rotatable optical fiber, optical core 110 (e.g. optical core 110 is positioned within lumen 1205), comprising a proximal end 1101 and a distal end 1109. Optical core 110 can comprise a dispersion shifted optical fiber, such as a depressed cladding dispersion shifted fiber (e.g. a Non-Zero Dispersion Shifted, NZDS, fiber). Shaft 120 further comprises a distal portion 1208, including a transparent window, window 130 (e.g. a window that is relatively transparent to the one or more frequencies of light transmitted through optical core 110). An optical assembly, optical assembly 115, is operably attached to the distal end 1109 of optical core 110. Optical assembly 115 is positioned within window 130 of shaft 120. Optical assembly 115 can comprise a GRIN lens optically coupled to the distal end 1109 of optical core 110. Optical assembly 115 can comprise a construction and arrangement similar to optical assembly 115 as described in applicant's co-pending U.S. patent application Ser. No. 16/764,087, titled “Imaging System”, filed May 14, 2020, and applicant's co-pending U.S. patent application Ser. No. 17/276,500, titled “Imaging System with Optical Pathway”, filed Mar. 16, 2021, the content of each of which is incorporated herein by reference in its entirety for all purposes. A connector assembly, connector assembly 150, is positioned on the proximal end of shaft 120. Connector assembly 150 operably attaches imaging probe 100 to rotation assembly 500, as described herein. Connector assembly 150 surrounds and operably attaches to an optical connector 161, fixedly attached to the proximal end of optical core 110. A second connector, pullback connector 180, is positioned on shaft 120. Connector 180 can be removably attached and/or adjustably positioned along the length of shaft 120. Connector 180 can be positioned along shaft 120, such as by a clinician, operator or other user of system 10 (“user” or “operator” herein), proximate the proximal end of delivery catheter 80 after imaging probe 100 has been inserted into a patient via delivery catheter 80. Shaft 120 can comprise a portion between connector assembly 150 and the placement location of connector 180 that accommodates slack in shaft 120, a proximal portion of shaft 120 (e.g. a proximal portion of imaging probe 100), service loop 185. In some embodiments, optical core 110 comprises a single length of optical fiber comprising zero splices along its length. In some embodiments, imaging probe 100 comprises a single optical splice, such as a splice being between optical assembly 115 and distal end 1109 of optical core 110 (e.g. when there are zero splices along the length of optical core 110).

In some embodiments, shaft 120 comprises a multi-part construction, such as an assembly of two or more tubes that can be connected in various ways. In some embodiments, one or more tubes of shaft 120 can comprise tubes made of polyethylene terephthalate (PET), such as when a PET tube surrounds the junction between two tubes (e.g. two portions of shaft 120) in an axial arrangement to create a joint between the two tubes. In some embodiments, one or more PET tubes are under tension after assembly (e.g. the tubes are longitudinally stretched when shaft 120 is assembled), such as to prevent or at least reduce the tendency of the PET tube to wrinkle while shaft 120 is advanced through a tortuous path. In some embodiments, one or more portions of shaft 120 include a coating comprising one, two, or more materials and/or surface modifying processes, such as to provide a hydrophilic coating or a lubricious coating. In some embodiments, one or more metal portions of shaft 120 (e.g. nickel-titanium portions) are surrounded by a tube (e.g. a polymer tube), such as to improve the adhesion of a coating to that portion of shaft 120.

Imaging probe 100 can comprise one or more visualizable markers along its length (e.g. along shaft 120), markers 131a-b shown (marker 131 herein). Marker 131 can comprise markers selected from the group consisting of: radiopaque markers; ultrasonically reflective markers; magnetic markers; ferrous material; and combinations of one or more of these. In some embodiments, marker 131 comprises a marker positioned at a location (e.g. a location within and/or at least proximate distal portion 1208) to assist a user of imaging system 10 in performing a pullback procedure (“pullback procedure” or “pullback” herein), such as to cause tip 119 to be positioned at a location distal to the proximal end of an implant after the pullback is completed (e.g. so that imaging probe 100 can be safely advanced through the implant after the pullback).

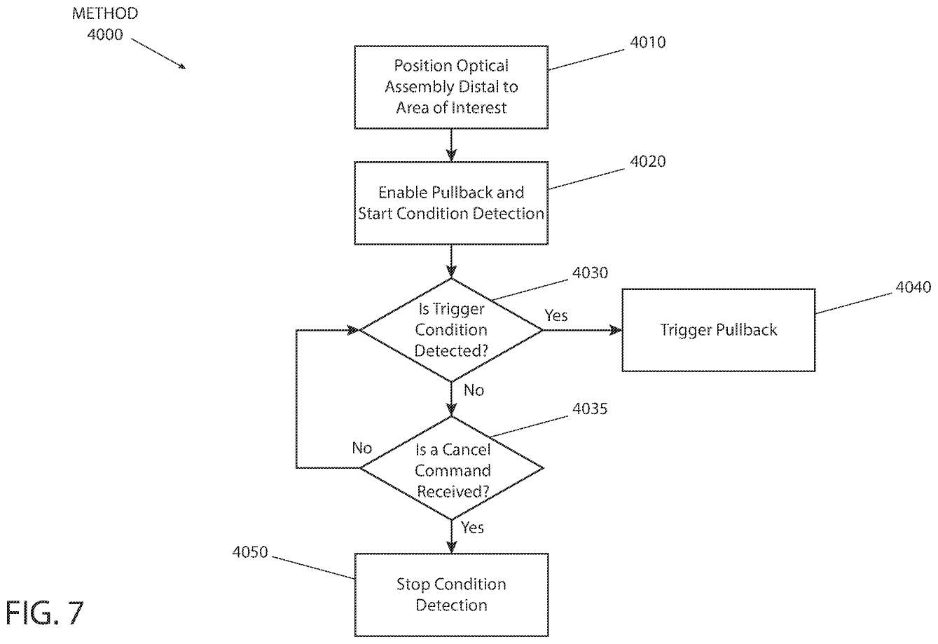

In some embodiments, system 10 is configured to perform a pullback procedure, and system 10 is further configured to initiate the pullback procedure based on an analysis of an electrocardiogram (EKG) signal, such as an EKG signal produced via an analysis of OCT data by algorithm 51 as described in reference to FIG. 7 and otherwise herein.

In some embodiments, imaging probe 100 includes a viscous dampening material, gel 118, positioned within shaft 120 and surrounding optical assembly 115 and a distal portion of optical core 110 (e.g. a gel injected or otherwise installed in a manufacturing process). Gel 118 can comprise a non-Newtonian fluid, for example a shear-thinning fluid. In some embodiments, gel 118 comprises a static viscosity of greater than 500 centipoise, and a shear viscosity that is less than the static viscosity. In these embodiments, the ratio of static viscosity to shear viscosity of gel 118 can be between 1.2:1 and 100:1. In some embodiments, gel 118 is injected from the distal end of window 130 (e.g. in a manufacturing process). In some embodiments, gel 118 comprises a gel which is visualizable under UV light (e.g. when gel 118 includes one or more materials that fluoresce under UV light). In some embodiments, during a manufacturing process when gel 118 is injected into shaft 120 via window 130, shaft 120 is monitored while being illuminated by UV light such that the injection process can be controlled (e.g. injection is stopped when gel 118 sufficiently ingresses into shaft 120). Gel 118 can comprise a gel as described in reference to applicant's co-pending U.S. patent application Ser. No. 15/566,041, titled “Micro-Optic Probes for Neurology”, filed Oct. 12, 2017, and applicant's co-pending U.S. patent application Ser. No. 16/764,087, titled “Imaging System”, filed May 14, 2020, the content of each of which is incorporated herein by reference in its entirety for all purposes.

Imaging probe 100 can include a distal tip portion, distal tip 119. In some embodiments, distal tip 119 can comprise a spring tip, such as a spring tip configured to improve the “navigability” of imaging probe 100 (e.g. to improve “trackability” and/or “steerability” of imaging probe 100), for example within a tortuous pathway (e.g. within a blood vessel or other vessel of the brain or heart with a tortuous pathway). In some embodiments, tip 119 comprises a length of between 5 mm and 100 mm (e.g. a spring with a length between 5 mm and 100 mm). In some embodiments, spring tip 119 can comprise a user shapeable spring tip (e.g. at least a portion of spring tip 119 is malleable). Imaging probe 100 can be rotated (e.g. via connector 180) to adjust the direction of a non-linear shaped portion of spring tip 119 (e.g. to adjust the trajectory of spring tip 119 in the vasculature of the patient). Alternatively or additionally, tip 119 can comprise a cap, plug, or other element configured to seal the distal opening of window 130. In some embodiments, tip 119 can comprise a radiopaque marker configured to increase the visibility of imaging probe 100 under an X-ray or fluoroscope. In some embodiments, tip 119 can comprise a relatively short luminal guidewire pathway to allow “rapid exchange” translation of imaging probe 100 over a guidewire of system 10 (guidewire not shown).

In some embodiments, at least the distal portion of imaging probe 100 (e.g. the distal portion 1208 of shaft 120 surrounding optical assembly 115) comprises an outer diameter of no more than 0.030″, such as no more than 0.025″, no more than 0.020″, and/or no more than 0.016″.

In some embodiments, imaging probe 100 can be constructed and arranged for use in an intravascular neural procedure (e.g. a procedure in which the blood, vasculature, and other tissue proximate the brain are visualized, and/or devices positioned temporarily or permanently proximate the brain are visualized). An imaging probe 100 configured for use in a neural procedure can comprise an overall length of at least 150 cm, such as a length of approximately 300 cm. Alternatively or additionally, imaging probe 100 can be constructed and arranged for use in an intravascular cardiac procedure (e.g. a procedure in which the blood, vasculature, and other tissue proximate the heart are visualized, and/or devices positioned temporarily or permanently proximate the heart are visualized). An imaging probe 100 configured for use in a cardiovascular procedure can comprise an overall length of at least 120 cm, such as an overall length of approximately 280 cm (e.g. to allow placement of the proximal end of probe 100 outside of the sterile field). In some embodiments, such as for placement outside of the sterile field, imaging probe 100 can comprise a length greater than 220 cm and/or less than 320 cm.

Rotation assembly 500 comprises a connector assembly 510, operably attached to a rotary joint 550. Rotation assembly 500 further comprises a motor or other rotational energy source, motive element 530. Motive element 530 is operably attached to rotary joint 550 via a linkage assembly 540. In some embodiments, linkage assembly 540 comprises one or more gears, belts, pulleys, or other force transfer mechanisms. Motive element 530 can drive (e.g. rotate via linkage assembly 540) rotary joint 550 (and in turn core 110) at speeds of at least 100 rotations per second, such as at least 200 rotations per second, 250 rotations per second, 400 rotations per second, 500 rotations per second, or between 20 rotations per second and 1000 rotations per second. Motive element 530 can comprise a mechanism selected from the group consisting of: a motor; a servo; a stepper motor (e.g. a stepper motor including a gear box); a linear actuator; a hollow core motor; and combinations thereof. In some embodiments, rotation assembly 500 is configured to rotate optical assembly 115 and rotatable optical core 110 in unison.

Connector assembly 510 operably attaches to connector assembly 150 of imaging probe 100, allowing optical connector 161 to operably engage rotary joint 550. In some embodiments, connector assembly 510 operably engages connector assembly 150. In some embodiments, connector assembly 510 operably engages connector assembly 150 such that rotary joint 550 and optical connector 161 are free to rotate within the engaged assemblies.

Retraction assembly 800 comprises a connector assembly 820, that operably attaches to a reference point, for example connector 82 of delivery catheter 80, such as to establish a reference for retraction assembly 800 relative to the patient. Connector assembly 820 can attach to a reference point such as a patient introduction device, surgical table, and/or another fixed or semi fixed point of reference. A retraction element, puller 850, releasably attaches to connector 180 of imaging probe 100, such as via a carrier 855. Retraction assembly 800 retracts at least a portion of imaging probe 100 (e.g. the portion of imaging probe 100 distal to the attached connector 180), relative to the established reference. In some embodiments, retraction assembly 800 is configured to retract at least a portion of imaging probe 100 (e.g. at least optical assembly 115 and a portion of shaft 120) at a rate of between 5 mm/sec and 100 mm/sec, such as 60 mm/sec. In some embodiments, retraction assembly 800 is configured to retract at least a portion of imaging probe 100 at a rate of at least 60 mm/sec, at least 80 mm/sec, at least 100 mm/sec, and/or at least 150 mm/sec. Additionally or alternatively, the pullback procedure can be performed during a time period of between 0.5 sec and 25 sec, for example approximately 20 sec (e.g. over a distance of 100 mm at 5 mm/sec). Service loop 185 of imaging probe 100 can be positioned between retraction assembly 800 and/or at least connector assembly 820, and rotation assembly 500, such that imaging probe 100 can be retracted relative to the patient while rotation assembly 500 remains stationary (e.g. attached to the surgical table and/or to a portion of console 50).

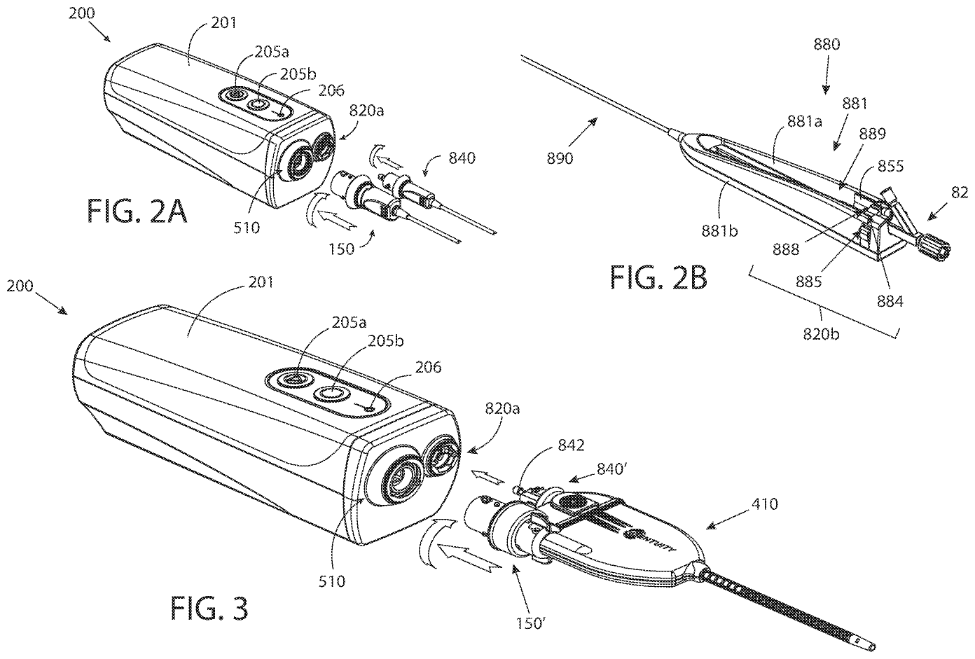

Retraction assembly 800 further comprises a linear drive, motive element 830. In some embodiments, motive element 830 can comprise a linear actuator, a worm drive operably attached to a motor, a pulley system, and/or other linear force transfer mechanisms. Puller 850 can be operably attached to motive element 830 via a linkage assembly 890. In some embodiments, linkage assembly 890 can comprise one or more components of a “pullback assembly”, as described in reference to FIGS. 1A and 2A. Alternatively or additionally, linkage assembly 890 can comprise one or more components of an enclosed pullback connector, as described in reference to FIG. 1B. One or more components of linkage assembly 890 can establish a frame of reference (e.g. an internal pullback reference) between puller 850 and the motive element 830, such that motive element 830 applies a pullback force to puller 850 via linkage assembly 890, and puller 850 retracts relative to the distal portion of linkage assembly 890 (e.g. relative to the distal end of sheath 895 as described in reference to FIG. 1A). In some embodiments, the distal end of linkage assembly 890 and connector assembly 820 are fixed relative to each other, and puller 850 translates linearly between the two in reaction to a force applied from motive element 830.

Console 50 comprises an imaging assembly 300, a user interface 55, processor 52, and one or more algorithms 51. Processor 52 can include one or more memory storage components, such as one or more memory circuits which store software routines, algorithms (e.g. algorithm 51), and other operating instructions of system 10, as well as data acquired by imaging probe 100, second imaging device 15, and/or another component of system 10. Imaging assembly 300 can be configured to provide light to optical assembly 115 (e.g. via optical core 110) and collect light from optical assembly 115 (e.g. via optical core 110). Imaging assembly 300 can include a light source 310. Light source 310 can comprise one or more light sources, such as one or more light sources configured to provide one or more wavelengths of light to optical assembly 115 via optical core 110. Light source 310 is configured to provide light to optical assembly 115 (via optical core 110) such that image data can be collected comprising cross-sectional, longitudinal and/or volumetric information related to a patient site or implanted device being imaged. Light source 310 can be configured to provide light such that the image data collected includes characteristics of tissue within the patient site being imaged, such as to quantify, qualify or otherwise provide information related to a patient disease or disorder present within the patient site being imaged. Light source 310 can be configured to deliver broadband light and have a center wavelength in the range from 350 nm to 2500 nm, from 800 nm to 1700 nm, from 1280 nm to 1310 nm, or approximately 1300 nm (e.g. light delivered with a sweep range from 1250 nm to 1350 nm). Light source 310 can comprise a sweep rate of at least 50 KHz. In some embodiments, light source 310 comprises a sweep rate of at least 100 KHz, such as at least 200 Khz, 300 KHz, 400 KHz, and/or 500 KHz. These faster sweep rates provide numerous advantages, such as to provide a higher frame rate, as well as being compatible with rapid pullback and rotation rates. For example, the higher sweep rate enables the requisite sampling density (e.g. the amount of luminal surface area swept by the rotating beam) to be achieved in a shorter time, advantageous in most situations and especially advantageous when there is relative motion between the probe and the surface/tissue being imaged such as arteries in a beating heart. Light source 310 bandwidth can be selected to achieve a desired resolution, which can vary according to the needs of the intended use of imaging system 10. In some embodiments, bandwidths are about 5% to 15% of the center wavelength, which allows resolutions of between 20 μm and 5 μm. Light source 310 can be configured to deliver light at a power level meeting ANSI Class 1 (“eye safe”) limits, though higher power levels can be employed. In some embodiments, light source 310 delivers light in the 1.3 μm band at a power level of approximately 20 mW. Tissue light scattering is reduced as the center wavelength of delivered light increases, however water absorption increases. Light source 310 can deliver light at a wavelength approximating 1300 nm to balance these two effects. Light source 310 can be configured to deliver shorter wavelength light (e.g. approximately 800 nm light) to traverse patient sites to be imaged including large amounts of fluid. Alternatively or additionally, light source 310 can be configured to deliver longer wavelengths of light (e.g. approximately 1700 nm light), such as to reduce a high level of scattering within a patient site to be imaged. In some embodiments, light source 310 comprises a tunable light source (e.g. light source 310 emits a single wavelength that changes repetitively over time), and/or a broad-band light source. Light source 310 can comprise a single spatial mode light source or a multimode light source (e.g. a multimode light source with spatial filtering).

Light source 310 can comprise a relatively long effective coherence length, such as a coherence length of greater than 10 mm, such as a length of at least 50 mm, at all frequencies within the bandwidth of the light source. This coherence length capability enables longer effective scan ranges to be achieved by system 10, as the light returning from distant objects to be imaged (e.g. tissue) must remain in phase coherence with the returning reference light, in order to produce detectable interference fringes. In the case of a swept-source laser, the instantaneous linewidth is very narrow (i.e. as the laser is sweeping, it is outputting a very narrow frequency band that changes at the sweep rate). Similarly, in the case of a broad-bandwidth source, the detector arrangement must be able to select very narrow linewidths from the spectrum of the source. The coherence length scales inversely with the linewidth. Longer scan ranges enable larger or more distant objects to be imaged (e.g. more distal tissue to be imaged). Current systems have lower coherence length, which correlates to reduced image capture range as well as artifacts (ghosts) that arise from objects outside the effective scan range.

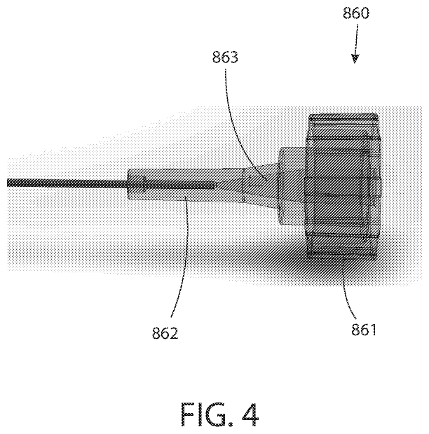

Console 50 can comprise one or more algorithms, such as algorithm 51 shown, which can be configured to adjust (e.g. automatically and/or semi-automatically adjust) one or more operational parameters of imaging system 10, such as an operational parameter of console 50, imaging probe 100 and/or a delivery catheter 80. Console 50 can further comprise a processing assembly, processor 52, configured to execute algorithm 51, and/or perform any type of data processing, such as digital signal processing, described in reference to FIG. 4. Additionally or alternatively, algorithm 51 can be configured to adjust an operational parameter of a separate device, such as injector 20 or implant delivery device 30 described herein. In some embodiments, algorithm 51 is configured to adjust an operational parameter based on one or more sensor signals, such as a sensor signal provided by a sensor-based functional element of the present inventive concepts as described herein. Algorithm 51 can be configured to adjust an operational parameter selected from the group consisting of: a rotational parameter such as rotational velocity of optical core 110 and/or optical assembly 115; a retraction parameter of shaft 120 and/or optical assembly 115 such as retraction velocity, distance, start position, end position and/or retraction initiation timing (e.g. when retraction is initiated); a position parameter such as position of optical assembly 115; a line spacing parameter such as lines per frame; an image display parameter such as a scaling of display size to vessel diameter; an imaging probe 100 configuration parameter; an injectate 21 parameter such as a saline to contrast ratio configured to determine an appropriate index of refraction; a light source 310 parameter such as power delivered and/or frequency of light delivered; and combinations of one or more of these. In some embodiments, algorithm 51 is configured to adjust a retraction parameter such as a parameter triggering the initiation of the pullback, such as a pullback that is initiated based on a parameter selected from the group consisting of: lumen flushing (the lumen proximate optical assembly 115 has been sufficiently cleared of blood or other matter that would interfere with image creation); an indicator signal is received from injector 20 (e.g. a signal indicating sufficient flushing fluid has been delivered); a change in image data collected (e.g. a change in an image is detected, based on the image data collected, that correlates to proper evacuation of blood from around optical assembly 115); and combinations of one or more of these. In some embodiments, algorithm 51 is configured to adjust an imaging system 10 configuration parameter related to imaging probe 100, such as when algorithm 51 identifies (e.g. automatically identifies via an RF identifier and/or other embedded identifier) the attached imaging probe 100 and adjusts an imaging system 10 parameter, such as an optical path length parameter, a dispersion parameter, and/or other parameter as listed above.

In some embodiments, algorithm 51 is configured to trigger the initiation of a pullback based on a time-gated parameter. In some embodiments, a T-wave trigger (e.g. provided by a separate device) can be provided to console 50 to begin pullback when the low-motion portion of the heart cycle is detected. As an alternative to a T-wave trigger, or in addition to it, motion patterns (e.g. relative motion patterns) can be tracked (e.g. using angiography) between one or more portions (e.g. components or other features) of probe 100 and relatively stable (e.g. non-moving) portions of the patient's anatomy (e.g. ribs, sternum and/or spinal column).

When a console 50 of system 10 is first installed at a clinical site (e.g. a catheter lab), a simple calibration routine can be used to establish the latency between the angiographic system and system 10. Essentially, a probe 100 is provided, an angiographic system at the clinical site is engaged and an angiographic image feed is provided to console 50 (e.g. using any standard video connection, analog or digital). Angiographic system-provided video frames are registered according to a clock of console 50, which is used as a reference time frame. A pullback (e.g. in a patient or in a non-patient simulation mode) of probe 100 is initiated (also coordinated by the console 50 clock) and captured by angiography. A trained user or technician reviews the angiographic image frames and designates the first frame in which motion was detected. This process establishes the associated latency according the console 50 clock. The motion detection can also be automated, for example using a neural network trained to recognize probe 100 movement (e.g. movement of a marker band of probe 100) under angiography.

In some embodiments, a calibration procedure to establish the latency between an angiographic system and system 10, and an imaging procedure performed during relatively low motion of a heart cycle, includes the following steps. In a first step, angiography is initiated once probe 100 has been inserted into the patient and deployed into the target anatomy. In a second step, system 10 analyzes the relative motion between one or more portions of probe 100 (e.g. motion of a marker band or other probe 100 portion which follows the beating heart of the patient) and more stable features in the image, such as images of the sternum or spinal column. Once a cardiac rhythm has been established and the low motion portion identified (typically 5-10 heart cycles are used for this analysis, which can be velocity vector analysis, neural network analysis, and the like), an indicator is provided and a system 10 “metronome” is started. System 10 can reference the output of the metronome, such as at the time that radiopaque flushing material is injected to clear the blood from the target area to be imaged, since the one or more portions of probe 100 (e.g. one or more marker bands) can become radio-invisible during this flushing period. In an alternative embodiment, a non-radiopaque flushing material can be used (e.g. dextran). In a third step, the flushing is started, such as by an operator or in automated way controlled by system 10. The flushing should continue over several heart cycles, such as 3-5 heart cycles. In a fourth step, clearing of the vessel to be imaged is detected by system 10 analyzing one or more of the images it produces. In a fifth step, at the low motion part of the metronome (e.g. a predicted low motion portion of the heart cycle), and accounting for the latency between system 10 and the angiographic system previously established, a pullback starts. In some embodiments, the pullback will finish in about one-half of a heart cycle or less, such as to remain within the low motion portion of the heart cycle. System 10 can be configured to provide a pullback speed of at least 50 mm/sec, such as at least 100 mm/sec, or 200 mm/sec. In a sixth step, the pullback sequence of images, which include minimal motion artifact, can be provided to the operator and/or used for CFD calculations, implant (e.g. stent) length measurements, and the like. The use of image capture during low motion, as described herein, avoids errors associated with motion artifacts, notably longitudinal motion artifacts.

In some embodiments, algorithm 51 is configured to perform one, two, or more analyses of the OCT data (e.g. filtering or other image processing analyses) that provide image stabilization (e.g. of displayed OCT data).

Imaging system 10 can comprise one or more interconnect cables, bus 58 shown. Bus 58 can operably connect rotation assembly 500 to console 50, retraction assembly 800 to console 50, and or rotation assembly 500 to retraction assembly 800. Bus 58 can comprise one or more optical transmission fibers, electrical transmission cables, fluid conduits, and combinations of one or more of these. In some embodiments, bus 58 comprises at least an optical transmission fiber that optically couples rotary joint 550 to imaging assembly 300 of console 50. Additionally or alternatively, bus 58 comprises at least power and/or data transmission cables that transfer power and/or motive information to one or more of motive elements 530 and 830.

Second imaging device 15 can comprise an imaging device such as one or more imaging devices selected from the group consisting of: an X-ray; a fluoroscope such as a single plane or biplane fluoroscope; a CT Scanner; an MRI; a PET Scanner; an ultrasound imager; and combinations of one or more of these. In some embodiments, second imaging device 15 comprises a device configured to perform rotational angiography.

Treatment device 16 can comprise an occlusion treatment or other treatment device selected from the group consisting of: a balloon catheter constructed and arranged to dilate a stenosis or other narrowing of a blood vessel or other vessel of the patient; a drug eluting balloon; an aspiration catheter; a sonolysis device; an atherectomy device; a thrombus removal device such as a stent retriever device; a Trevo™ stentriever; a Solitaire™ stentriever; a Revive™ stentriever; an Eric™ stentriever; a Lazarus™ stentriever; a stent delivery catheter; a microbraid implant; an embolization system; a WEB™ embolization system; a Luna™ embolization system; a Medina™ embolization system; and combinations of one or more of these. In some embodiments, imaging probe 100 is configured to collect data related to treatment device 16 (e.g. treatment device 16 location, orientation and/or other configuration data), after treatment device 16 has been inserted into the patient.

Patient monitoring device 17 can comprise one or more monitoring devices selected from the group consisting of: an ECG monitor; an EEG monitor; a blood pressure monitor; a blood flow monitor; a respiration monitor; a patient movement monitor; a T-wave trigger monitor; and combinations of these.

Injector 20 can comprise a power injector, syringe pump, peristaltic pump or other fluid delivery device configured to inject a contrast agent, such as radiopaque contrast, and/or other fluids. In some embodiments, injector 20 is configured to deliver contrast and/or other fluid (e.g. contrast, saline and/or Dextran). In some embodiments, injector 20 delivers fluid in a flushing procedure as described herein. In some embodiments, injector 20 delivers contrast or other fluid through a delivery catheter 80 with an ID of between 5Fr and 9Fr, a delivery catheter 80 with an ID of between 0.53″ to 0.70″, or a delivery catheter 80 with an ID between 0.0165″ and 0.027″. In some embodiments, contrast or other fluid is delivered through a delivery catheter as small as 4Fr (e.g. for distal injections). In some embodiments, injector 20 delivers contrast and/or other fluid through the lumen of one or more delivery catheters 80, while one or more smaller delivery catheters 80 also reside within the lumen. In some embodiments, injector 20 is configured to deliver two dissimilar fluids simultaneously and/or sequentially, such as a first fluid delivered from a first reservoir and comprising a first concentration of contrast, and a second fluid from a second reservoir and comprising less or no contrast.

Injectate 21 can comprise fluid selected from the group consisting of: optically transparent material; saline; visualizable material; contrast; Dextran; an ultrasonically reflective material; a magnetic material; and combinations thereof. Injectate 21 can comprise contrast and saline. Injectate 21 can comprise at least 20% contrast. During collection of image data, a flushing procedure can be performed, such as by delivering one or more fluids, injectate 21 (e.g. as propelled by injector 20 or other fluid delivery device), to remove blood or other somewhat opaque material (hereinafter non-transparent material) proximate optical assembly 115 (e.g. to remove non-transparent material between optical assembly 115 and a delivery catheter and/or non-transparent material between optical assembly 115 and a vessel wall), such as to allow light distributed from optical assembly 115 to reach and reflectively return from all tissue and other objects to be imaged. In these flushing embodiments, injectate 21 can comprise an optically transparent material, such as saline. Injectate 21 can comprise one or more visualizable materials, as described herein.

As an alternative or in addition to its use in a flushing procedure, injectate 21 can comprise material configured to be viewed by second imaging device 15, such as when injectate 21 comprises a contrast material configured to be viewed by a second imaging device 15 comprising a fluoroscope or other X-ray device; an ultrasonically reflective material configured to be viewed by a second imaging device 15 comprising an ultrasound imager; and/or a magnetic material configured to be viewed by a second imaging device 15 comprising an Mill.

Implant 31 can comprise an implant (e.g. a temporary or chronic implant) for treating one or more of a vascular occlusion or an aneurysm. In some embodiments, implant 31 comprises one or more implants selected from the group consisting of: a flow diverter; a Pipeline™ flow diverter; a Surpass™ flow diverter; an embolization coil; a stent; a Wingspan™ stent; a covered stent; an aneurysm treatment implant; and combinations of one or more of these.

Implant delivery device 30 can comprise a catheter or other tool used to deliver implant 31, such as when implant 31 comprises a self-expanding or balloon expandable portion. In some embodiments, imaging system 10 comprises imaging probe 100, one or more implants 31 and/or one or more implant delivery devices 30. In some embodiments, imaging probe 100 is configured to collect data related to implant 31 and/or implant delivery device 30 (e.g. implant 31 and/or implant delivery device 30 anatomical location, orientation and/or other configuration data), after implant 31 and/or implant delivery device 30 has been inserted into the patient.

In some embodiments, one or more system components, such as console 50, delivery catheter 80, imaging probe 100, rotation assembly 500, retraction assembly 800, treatment device 16, injector 20, and/or implant delivery device 30, further comprise one or more functional elements (“functional element” herein), such as functional elements 59, 89, 199, 599, 899, 99a, 99b, and/or 99c, respectively, shown. Each functional element can comprise at least two functional elements. Each functional element can comprise one or more elements selected from the group consisting of: sensor; transducer; and combinations thereof. The functional element can comprise a sensor configured to produce a signal. The functional element can comprise a sensor selected from the group consisting of: a physiologic sensor; a pressure sensor; a strain gauge; a position sensor; a GPS sensor; an accelerometer; a temperature sensor; a magnetic sensor; a chemical sensor; a biochemical sensor; a protein sensor; a flow sensor such as an ultrasonic flow sensor; a gas detecting sensor such as an ultrasonic bubble detector; a sound sensor such as an ultrasound sensor; and combinations thereof. The sensor can comprise a physiologic sensor selected from the group consisting of: a pressure sensor such as a blood pressure sensor; a blood gas sensor; a flow sensor such as a blood flow sensor; a temperature sensor such as a blood or other tissue temperature sensor; and combinations thereof. The sensor can comprise a position sensor configured to produce a signal related to a vessel path geometry (e.g. a 2D or 3D vessel path geometry). The sensor can comprise a magnetic sensor. The sensor can comprise a flow sensor. The system can further comprise an algorithm configured to process the signal produced by the sensor-based functional element. Each functional element can comprise one or more transducers. Each functional element can comprise one or more transducers selected from the group consisting of: a heating element such as a heating element configured to deliver sufficient heat to ablate tissue; a cooling element such as a cooling element configured to deliver cryogenic energy to ablate tissue; a sound transducer such as an ultrasound transducer; a vibrational transducer; and combinations thereof.

In some embodiments, imaging probe 100 comprises a fluid propulsion element and/or a fluid pressurization element (“fluid pressurization element” herein), FPE 1500. FPE 1500 can be configured to prevent and/or reduce the presence of bubbles within gel 118 proximate optical assembly 115. FPE 1500 can be fixedly attached to optical core 110, wherein rotation of optical core 110 in turn rotates the fluid propulsion element, such as to generate a pressure increase within gel 118 that is configured to reduce presences of bubbles from locations proximate optical assembly 115. Such one or more fluid pressurization elements FPE 1500 can reduce the likelihood of bubble formation within gel 118, reduce the size of bubbles within gel 118, and/or move any bubbles formed within gel 118 away from a location that would adversely impact the collecting of image data by optical assembly 115 (e.g. move bubbles away from optical assembly 115). In some embodiments, a fluid propulsion element FPE 1500 of imaging probe 100 comprises a similar construction and arrangement to a fluid propulsion element described in applicant's co-pending International PCT Patent Application Serial Number PCT/US2020/030616, titled “Imaging Probe with Fluid Pressurization Element”, filed Apr. 30, 2020, the content of which is incorporated herein by reference in its entirety for all purposes.

In some embodiments, imaging probe 100 comprises an overall length of at least 120 cm, such as at least 160 cm, such as approximately 280 cm. In some embodiments, imaging probe 100 comprises an overall length of no more than 350 cm. In some embodiments, imaging probe 100 comprises a length configured to be inserted into the patient (“insertable length” herein) of at least 90 cm, such as at least 100 cm, such as approximately 145 cm. In some embodiments, imaging probe 100 comprises an insertable length of no more than 250 cm, such as no more than 200 cm. In some embodiments, tip 119 comprises a spring tip with a length of at least 5 mm, such as at least 25 mm, such as approximately 15 mm. In some embodiments, tip 119 comprises a spring tip with a length of no more than 75 mm, such as no more than 30 mm. In some embodiments, a distal portion of shaft 120 (e.g. window 130) comprises an outer diameter of less than 2Fr, such as less than 1.4Fr, such as approximately 1.1Fr. In some embodiments, a distal portion of shaft 120 (e.g. window 130) comprises an outer diameter of at least 0.5Fr, such as at least 0.9Fr. In some embodiments, shaft 120 comprises one or more materials selected from the group consisting of: polyether ether ketone (PEEK); nylon; polyether block amide; nickel-titanium alloy; and combinations of these.

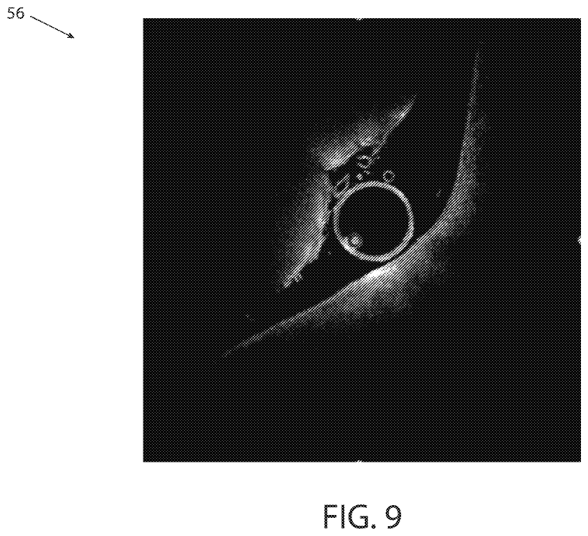

In some embodiments, at least a portion of imaging probe 100 (e.g. the most flexible portion) comprises a minimum radius of curvature of less than 5 mm, such as less than 4 mm, such as less than 3 mm, such as less than 2 mm, such as approximately 1 mm. In some embodiments optical core 110 comprises an optical fiber with a diameter of less than 120 μm, such as less than 100 μm, such as less than 80 μm, such as less than 60 μm, such as approximately 40 μm. In some embodiments, optical core 110 comprises a numerical aperture of one or more of 0.11, 0.14, 0.16, 0.17, 0.18, 0.20, and/or 0.25. In some embodiments, optical assembly 115 comprises a lens selected from the group consisting of: a GRIN lens; a molded lens; a shaped lens, such as a melted and polished lens; a lens comprising an axicon structure, (e.g. an axicon nano-structure); and combinations of these. In some embodiments, optical assembly 115 comprises a lens with an outer diameter of less than 200 μm, such as less than 170 μm, such as less than 150 μm, such as less than 100 μm, such as approximately 80 μm. In some embodiments optical assembly 115 comprises a lens with a length of less than 3 mm, such as less than 1.5 mm. In some embodiments, optical assembly 115 comprises a lens with a length of at least 0.5 mm, such as at least 1 mm. In some embodiments, optical assembly 115 comprises a lens with a focal length of at least 0.5 mm and/or no more than 5.0 mm, such as at least 1.0 mm and/or no more than 3.0 mm, such as a focal length of approximately 0.5 mm. In some embodiments, optical assembly 115 can comprise longer focal lengths, such as to view structures outside of the blood vessel or other vessel in which optical assembly 115 is inserted, such as is described herebelow in reference to FIG. 9. In some embodiments, optical assembly 115 has a working distance (also termed depth of field, confocal distance, or Rayleigh Range) of up to 1 mm, such as up to 5 mm, such as up to 10 mm, such as a working distance of at least 1 mm and/or no more than 5 mm. In some embodiments, optical assembly 115 comprises an outer diameter of at least 80 μm and/or no more than 200 μm, such as at least 150 μm and/or no more than 170 μm, such as an outer diameter of approximately 150 μm. In some embodiments, system 10 (e.g. retraction assembly 800) is configured to perform a pullback of probe 100 at a speed of at least 10 mm/sec and/or no more than 300 mm/sec, such as at least 50 mm/sec and/or no more than 200 mm/sec, such as a pullback speed of approximately 100 mm/sec. In some embodiments, system 10 (e.g. retraction assembly 800) is configured to perform a pullback for a distance of at least 25 mm and/or no more than 200 mm, such as at least 25 mm and/or no more than 150 mm, such as a distance of approximately 50 mm. In some embodiments, system 10 (e.g. retraction assembly 800) is configured to perform a pullback over a time period of at least 0.2 seconds and/or no more than 5.0 seconds, such as at least 0.5 seconds and/or no more than 2.0 seconds, such as a time period of approximately 1.0 second. In some embodiments, system 10 (e.g. rotation assembly 500) is configured to rotate optical core 110 at an angular velocity of at least 20 rotations per second and/or no more than 1000 rotations per second, such as at least 100 rotations per second and/or no more than 500 rotations per second, such as an angular velocity of approximately 250 rotations per second. In some embodiments, delivery catheter 80 comprises an inner diameter of at least 0.016″ and/or no more than 0.050″, such as at least 0.016″ and/or no more than 0.027″, such as an inner diameter of approximately 0.021″. In some embodiments, light source 310 comprises a sweep rate of at least 20 kHz and/or no more than 2000 kHz, such as at least 50 kHz and/or no more than 500 kHz, such as a sweep rate of approximately 200 kHz. In some embodiments, light source 310 comprises a sweep bandwidth of at least 30 nm and/or no more than 250 nm, such as at least 50 nm and/or no more than 150 nm, such as a sweep bandwidth of approximately 100 nm. In some embodiments, light source 310 comprises a center wavelength of at least 800 nm and/or no more than 1800 nm, such as at least 1200 nm and/or no more than 1350 nm, such as a center wavelength of approximately 1300 nm. In some embodiments, light source 310 comprises an optical power of at least 5 mW and/or no more than 500 mW, such as at least 10 mW and/or no more than 50 mW, such as an optical power of approximately 20 mW.

Referring now to FIG. 1A, a schematic view of an imaging system is illustrated, the system comprising an imaging probe operably attachable to a patient interface module, and an independent pullback module operably attachable to the patient interface module and the imaging probe, consistent with the present inventive concepts. Imaging system 10 can comprise a patient interface module 200. Patient interface module 200 comprises a housing, housing 201, surrounding at least a portion of rotation assembly 500, and at least a portion of retraction assembly 800. Imaging system 10 can further comprise a second, discrete component, pullback module 880. Pullback module 880 comprises a housing, housing 881, surrounding at least a portion of retraction assembly 800. Pullback module 880 and patient interface module 200 can be operably attached to each other via a connector assembly, linkage assembly 890 described herein. Pullback module 880 and patient interface module 200 can be constructed and arranged (via each having a separate housing) to enable positioning at different locations (e.g. linkage assembly 890 connecting modules 880 and 200 can comprise a length of at least 15 cm such that the two remote locations can be at least 15 cm apart), for example patient interface module 200 can be positioned on or near a surgical bed rail, and pullback module 880 can be positioned near a vascular access site of the patient (e.g. within 30 cm of the vascular access site thru which imaging probe 100 enters the patient). Linkage assembly 890 can comprise a linkage 891 slidingly received within sheath 895. Linkage 891 is operably attached to puller 850, and the proximal end 893 of linkage 891 can comprise a connection point, 842. Motive element 830 can comprise a connector 835 which can be configured to releasably attach to connection point 842. Components shown in FIG. 1A can be of similar construction and arrangement to like components described in reference to FIG. 1, and as described elsewhere herein.

Pullback module 880 can comprise a connector assembly 820b that operably attaches to connector 82 of delivery catheter 80, such as described in reference to FIG. 2B. Connector assembly 845 can comprise a connector 840 that operably attaches to a connector assembly 820a of patient interface module 200, as described in reference to FIG. 2A.

Referring now to FIG. 1B, a schematic view of an imaging system is illustrated, the system comprising an imaging probe operably attachable to a module comprising a first connector for attaching to a rotation motive element and a second connector for attaching to a retraction motive element, consistent with the present inventive concepts. Imaging system 10 can comprise a patient interface module 200 as described herein. Imaging system 10 can further comprise a connector module, module 410. Module 410 comprises a housing, housing 411, surrounding at least a portion of retraction assembly 800, service loop 185 of imaging probe 100, connector assembly 150′, and connector 840′. Module 410 can be configured to operably attach both imaging probe 100 and a linkage, puller 850′, to patient interface module 200. Components shown in FIG. 1B can be of similar construction and arrangement to like components described in reference to FIG. 1, and as described elsewhere herein. Module 410 can be operably attached to a delivery catheter 480. Delivery catheter 480 can be of similar construction and arrangement to delivery catheter 80 described in reference to FIG. 1. Delivery catheter 480 can comprise at least a portion that is optically transparent, window 485. Window 485 can be positioned at or near a distal portion of delivery catheter 480. Window 485 can comprise a material transparent to imaging modalities utilized by imaging probe 100, such that imaging probe 100 can image through window 485, for example when optical assembly 115 is retracted within window 485. In some embodiments, module 410, delivery catheter 480, and imaging probe 100 collectively form catheter assembly 490.

Referring now to FIG. 2A, a perspective view of connectors being attached to a patient interface is illustrated, consistent with the present inventive concepts. Patient interface module 200 is configured to provide rotation to a rotatable optical core of an imaging probe, and to provide a motive force to translate at least a portion of the imaging probe, such as is described herein. Patient interface module 200 comprises rotation assembly 500, and at least a portion of retraction assembly 800. A housing 201 surrounds patient interface module 200. Patient interface module 200 can comprise one or more user interface elements, such as one or more inputs, buttons 205a,b, and one or more outputs, indicator 206 shown. Patient interface module 200 comprises a first physical connector assembly, connector assembly 510, for operably connecting to connector assembly 150 as described herein. Patient interface module 200 can further comprise a second physical connector assembly, connector assembly 820a, for operably connecting to connector 840 also as described herein. Connector assembly 150 and connector 840 can each comprise bayonet type connectors, constructed and arranged to be at least partially inserted into connector assemblies 510 and 820a, respectively. Connector assembly 150 and connector 840 can be subsequently rotated (e.g. an approximately 45° rotation) to lock their connections with connector assemblies 510 and 820a, respectively, as described herein. Connector assembly 150 and/or connector 840 can comprise numerous forms of connectors, such as a bayonet or other locking connectors.

Referring now to FIG. 2B, a perspective view of a pullback assembly is illustrated, consistent with the present inventive concepts. Pullback module 880 can be operably attached to a portion of an imaging probe 10 of the present inventive concepts, and provide a retraction force to the probe, pulling at least a portion of the probe proximally relative to a patient (e.g. relative to a patient introduction device), as described herein. Pullback module 880 can comprise a construction and arrangement similar to pullback module 880 as described in applicant's co-pending U.S. patent application Ser. No. 16/764,087, titled “Imaging System”, filed May 14, 2020, the content of which is incorporated herein by reference in its entirety. Pullback module 880 can be operably attached to the distal end of a linkage 891 (not shown). Linkage assembly 890 can be slidingly received through pullback module 880. Sheath 895 can be fixedly attached to the proximal end of module 880. Linkage 891 is slidingly received along the length of module 880 and is operably attached at its distal end to puller 850.

Pullback module 880 can comprise a two-part housing 881, including a top housing 881a and bottom housing 881b. Module 880 can contain a translating cart, puller 850 (not shown, but positioned below carrier 855, and as described herein). Puller 850 can be designed to translate within module 880. Module 880 can comprise a biasing element, spring 852 (not shown). Spring 852 can provide a biasing force to puller 850, such as to bias puller 850 distally.

Top housing 881a can comprise a first cavity, retention port 884 and a second cavity, trench 889. Retention port 884 and trench 889 can be separated by a projection, retention wall 888. Physical connector assembly 820b can comprise a retention port 884 of housing 881a, including wall 888, and a retention mechanism, clip 885. Clip 885 can be configured to releasably engage the proximal end of a delivery catheter such as sheath connector 82 of delivery catheter 80, such as when connector 82 comprises a Tuohy Borst connector. Physical connector assembly 820b can further comprise a biasing element, spring 886 (not shown). Spring 886 can provide a biasing force to maintain clip 885 in an engaged position about connector 82.

Pullback module 880 can further comprise a carrier 855. Carrier 855 can operably attach to puller 850, such as through a slot in housing 881a. Carrier 855 can translate within trench 889 in response to puller 850, which translates in response to linkage 891. Carrier 855 can operably attach to a portion of imaging probe 100, such as to a pullback connector 180. Pullback connector 180 can comprise a “torquer”, or other device affixed to shaft 120 of imaging probe 100. Sheath 895 of linkage assembly 890 can provide a frame of reference between connector 840 and pullback module 880, such that when the proximal end of linkage 891 is retracted relative to connector 840, the distal end of linkage 891 is retracted towards sheath 895 (i.e. towards the proximal end of pullback module 880). This relative motion transfers motive force applied at connector 840 (e.g. via motive element 830, as described herein), to puller 850. Puller 850 subsequently transfers the motive force to imaging probe 100, and imaging probe 100 is retracted relative to the patient.

In operation, imaging probe 100 can be manually (e.g. by a clinician of the patient) advanced through the vasculature of the patient. Pullback module 880 can be attached to the patient (e.g. to delivery catheter 80 via connector 82), and connector 180 can be operably connected to imaging probe 100 and positioned proximate delivery catheter 80 (e.g. a torquer connector 180 can be tightened to imaging probe 100 proximate delivery catheter 80). Connector 180 (not shown) can be operably positioned within carrier 855, and a motive force can be applied to the distal end of linkage 891. Carrier 855 retracts within trench 889, retracting imaging probe 100 relative to the patient. After retraction, connector 180 can be removed from carrier 855 (e.g. lifted out of), and carrier 855 and imaging probe 100 can be re-advanced independently. For example, carrier 855 can re-advance via the bias of spring 852, as the proximal end of linkage 891 is allowed to advance, and imaging probe 100 can be re-advanced manually by an operator of system 10. Subsequent retractions can be performed by repositioning connector 180 in carrier 855 after both have been re-advanced. Carrier 855 can comprise a capturing portion, such as a “cup-like” geometry, a hook, or other capture-enabling portion, such that carrier 855 can only impart a retraction force on connector 180. In this configuration, if carrier 855 were to translate distally, connector 180 would automatically disengage from carrier 855 (e.g. connector 180 would fall out of the cup portion of carrier 855).

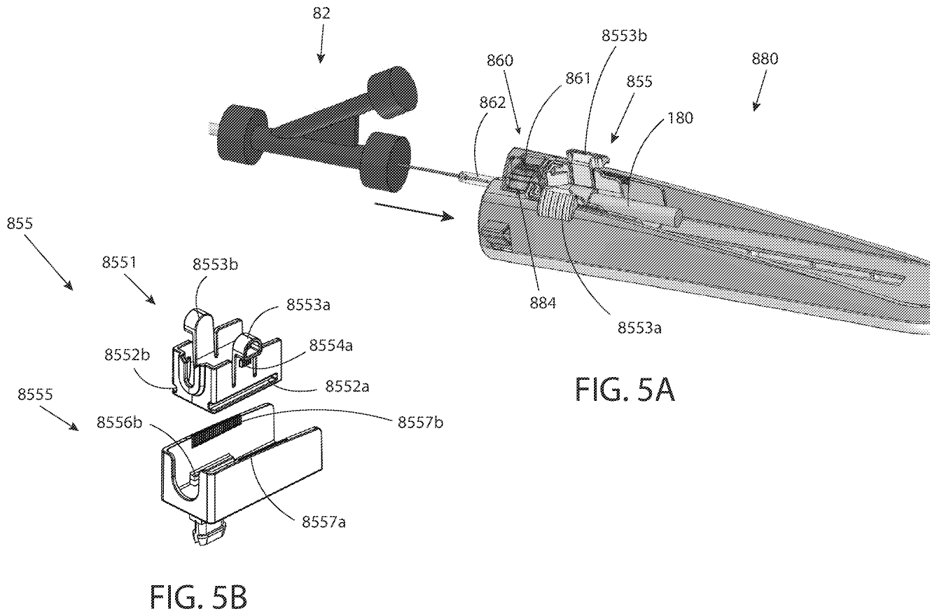

In some embodiments, carrier 855 comprises a construction and arrangement similar to carrier 855 described in reference to FIGS. 5A and 5B herein. As illustrated in FIGS. 5A and 5B, carrier 855 can comprise a two-piece assembly which enables micro-adjustability of the carrier 855 to accommodate variations in the positioning of pullback connector 180 relative to delivery catheter 80. The adjustability of the two-piece assembly is laterally constrained but is allowed to be adjusted axially. Carrier 855 can comprise one or more user graspable projections, and one or more toothed features on a first portion of the two-piece assembly that engage with notched features on a second portion of the two-piece assembly, thereby locking the components together during use. By depressing the projections, carrier 855 can be adjusted and locked into a new position.

Referring now to FIG. 3, a perspective view of connectors being attached to a patient interface module is illustrated, consistent with the present inventive concepts. Patient interface module 200 can be of similar construction and arrangement to patient interface module 200 as described in reference to FIG. 2A. Patient interface module 200 comprises a first physical connector assembly, connector assembly 510, for operably connecting to connector assembly 150′. Patient interface module 200 can further comprise a second physical connector assembly, connector assembly 820a, for operably connecting to connector 840′. Connector assembly 150′ and connector 840′ can each comprise bayonet type connectors, constructed and arranged to be at least partially inserted into connector assemblies 510 and 820a, respectively.

As described herein, system 10 can be constructed and arranged to provide improved imaging of a patient's anatomy (e.g. of one or more blood vessels or other vessels of the patient) as well as improved imaging of implants, catheters, and/or other devices positioned in the patient (e.g. positioned in a blood vessel or other vessel of the patient). In some embodiments, system 10 is configured to provide information that is used (e.g. by a clinician) to perform a treatment (e.g. an intervention), wherein the information is based on, at least, optical coherence tomography data. For example, OCT and other data gathered by system 10, can be used to plan a treatment and/or predict a treatment outcome (e.g. the planning and/or predicting performed by system 10, an operator of system 10, or a combination of the two), such as to impact a treatment to be delivered to the patient (“OCT-guided treatment” and/or “OCT-guided therapy” herein).

As described herein, imaging probe 100 can comprise at least one of: size (e.g. diameter and/or length), scan range, flexibility, and/or imaging capability configured to provide the improved imaging. Imaging probe 100 can comprise a size and/or flexibility configured to enable imaging of tight lesions within the vessel. As used herein, a tight lesion can comprise a lesion whose resultant lumen (i.e. the lumen within the lesion) comprises a diameter (e.g. the smallest diameter along the length of the lesion) of less than 2 mm (0.080″). A commercially available OCT catheter positioned to image a lesion with a lumen of this small diameter would effectively block the proximally-applied flush media from propagating to locations distal to the lesion, preventing the use of this commercial device. However, imaging probe 100 can be constructed and arranged to image these tight lesions, for example lesions with a resultant lumen as small as 1.5 mm (0.060″), as small as 1.3 mm (0.053″), as small as 1.1 mm (0.043″), and/or as small as 0.9 mm (0.036″), can be imaged by imaging probe 100. For example, the distal portion of imaging probe 100 can comprise an outer diameter of no more than 2.6Fr (0.034″), such as an outer diameter of no more than 1.7Fr (0.022″), such as to enable system 10 to be used to image potential vessels (e.g. arteries) to be treated that have a tight lesion, such as when the distal portion of imaging probe 100 is inserted into and through a stenosis, such as in a “pre-treatment” imaging procedure (e.g. a procedure performed prior to intervention or other treatment of the stenosis). As described herein, currently available OCT imaging systems can be too large to provide useful data (e.g. unable to pass thru and/or provide sufficient blood clearing in a tight lesion). Other types of imaging systems, such as angiography, may not provide sufficiently accurate results when imaging tight lesions (e.g. erroneously indicate no treatment is warranted, such as when providing FFR information). In some embodiments, system 10 is used to perform a pre-treatment imaging procedure (e.g. of a tight lesion) to gather data to enable OCT-guided treatment in which the data provided by system 10 (e.g. using images from at least probe 100) is used by an operator (e.g. a clinician) to make decisions about a future treatment to be performed. In these embodiments, system 10 can also be used to image a similar anatomical location, after the treatment has been performed (in a “post-treatment” imaging procedure).

In some embodiments, system 10 is configured to perform a pre-treatment imaging procedure (e.g. of a tight lesion or otherwise) and provide OCT-guided treatment due to the following characteristics of system 10: distal portion of probe 100 (e.g. including optical assembly 115) comprises a diameter of less than 2.6Fr (0.034″), such as a diameter of no more than 2.0Fr (0.026″), such as a diameter of no more than 1.7Fr (0.022″)

In some embodiments, system 10 is configured to perform a pre-treatment imaging procedure (e.g. of a tight lesion or otherwise) and provide OCT-guided treatment due to the following characteristics of system 10: optical assembly 115 is rotated (e.g. via rotation assembly 500) at a rate of more than 180 rotations per second, such as a rate of at least 200, 250, 400, and/or 500 rotations per second.

In some embodiments, system 10 is configured to perform a pre-treatment imaging procedure (e.g. of a tight lesion or otherwise) and provide OCT-guided treatment due to the following characteristics of system 10: scan range of system 10 is at least a radius of 7 mm, such as a radius of at least 11 mm. The long scan range of system 10 provides numerous advantages, such as the ability to image from the imaged vessel into any side branches of that vessel, the ability to image large vessels when optical assembly 115 is eccentrically positioned within the vessel lumen (e.g. proximate a portion of the vessel wall), and/or the ability to image larger vessels in general, such as the left main artery, carotid arteries, and large peripheral arteries.

In some embodiments, system 10 is configured to perform a pre-treatment imaging procedure (e.g. of a tight lesion or otherwise) and provide OCT-guided treatment due to the following characteristics of system 10: pullback distance of more than 7.5 cm, such as a pullback of at least 10 cm, or at least 15 cm. The pullback can be performed at a rate of at least 25 mm/sec, and/or within a time period of no more than 4 seconds (e.g. a complete pullback of at least 7.5 cm, 10 cm, and/or 15 cm in no more than 4 seconds). The operable pullback speed of imaging probe 100 can be determined via a relationship between the rotation rate of optical assembly 115 and the desired frame density (e.g. frames/mm) of the OCT image data, such that the pullback speed comprises the rotation rate divided by the frame density. Imaging probe 100 can comprise a rotation rate of greater than 180 Hz, such as at least 200 Hz or at least 250 Hz. Imaging probe 100 can comprise a frame spacing of no more than 0.2 mm (i.e. a frame density of at least 5 frames/mm). Imaging probe 100 can comprise a laser scan frequency of at least 200 KHz.

In some embodiments, system 10 is configured to perform a pre-treatment imaging procedure (e.g. of a tight lesion or otherwise) and provide OCT-guided treatment due to the following characteristics of system 10: pullback speed (the translation rate of optical assembly 115 during a pullback) of at least 50 mm/sec. In these embodiments, rotation rate of optical assembly 115 can be at least 180 Hz, 200 Hz, and/or 250 Hz. In these embodiments, the frame spacing can be 0.2 mm minimum.

In some embodiments, system 10 is configured to perform a pre-treatment imaging procedure (e.g. of a tight lesion or otherwise) and provide OCT-guided treatment due to the following characteristics of system 10: lines per frame of at least 400, such as at least 800 lines/frame, where a frame comprises approximately 360° of continuous image data (i.e. one full rotation of optical assembly 115 provides one frame of image data). In some embodiments, system 10 is configured to capture frames at a rate sufficient to allow down-sampling of the frames (e.g. down sampling performed prior to analog to digital conversion of the data, and/or other bandwidth-limited data processing).

In some embodiments, system 10 is configured to perform a pre-treatment imaging procedure (e.g. of a tight lesion or otherwise) and provide OCT-guided treatment due to the following characteristics of system 10: scan frequency of at least 50 kHz, such as at least 200 kHz, 350 kHz, and/or 500 kHz. In these embodiments, the lines per frame can be at least 400 lines/frame, or at least 800 lines/frame (e.g. where lines per frame equals the scan frequency divided by the rotation rate of optical assembly 115).

In some embodiments, system 10 comprises a laser scan frequency of no less than 200 kHz, a pullback speed of no less than 60 mm/sec or 100 mm/sec, and/or a rotation rate of no less than 250 Hz. System 10 can be configured to allow imaging of at least 50 mm of a vessel, such as at least 50 mm imaged in no more than 0.5 seconds, with no less than 800 scan lines per rotation, with approximately 400 μm pitch and/or a frame density of at least 2.5 frames/mm, and/or at least 5.0 frames/mm. In some embodiments, system 10 is configured to perform a pullback during a resting portion of the heart cycle to minimize motion artifacts. In some embodiments, system 10 comprises a rotation rate of up to 400 kHz, such as no less than 250 kHz, 300 kHz, or 350 kHz.

In some embodiments, system 10 is configured to perform a pre-treatment imaging procedure (e.g. of a tight lesion or otherwise) and provide OCT-guided treatment due to the following characteristics of system 10: processor 52 is configured to identify (e.g. via algorithm 51) a reflection generated at the splice interface between optical assembly 115 and optical core 110. The optical interface between optical assembly 115 (e.g. optical assembly 115 comprising a GRIN lens) and optical core 110 (e.g. optical core 110 comprising a NZDS fiber) can comprise a relatively large index mismatch, providing a clearly differentiable reflection. This reflection can provide a reference point for the OCT image data collected by system 10. In some embodiments, the interface can be identified by algorithm 51 with or without rotating optical core 110.

Referring now to FIG. 4, a perspective view of an adaptor for use with an imaging assembly is illustrated, consistent with the present inventive concepts. Retraction assembly 800 can include an interface, adapter 860. Adapter 860 can be constructed and arranged to simplify the insertion and/or connection of imaging probe 100 to connector 82. Adapter 860 comprises a hub 861, and a projection 862 extending distally from hub 861. Adapter 860 comprises a conduit 863 extending axially therethrough. In some embodiments, conduit 863 comprises a tapered conduit, such as a tapered conduit which tapers distally as shown. The proximal end of conduit 863 can comprise a diameter at least two times greater than the distal end of conduit 863. In some embodiments, projection 862 is constructed and arranged to be slidingly and lockingly received by a portion of connector 82 (e.g. when connector 82 comprises a Touhy-Borst type connector configured to lockingly engage projection 862). In some embodiments, hub 861 is constructed and arranged to be positioned within retention port 884 of pullback module 880, similar to that which is described in reference to FIG. 2B and to FIGS. 5A and 5B herein. In some embodiments, imaging probe 100 is slidingly positioned within conduit 863 prior to a clinical procedure (e.g. in a manufacturing and/or packaging process), such as to simplify one or more steps of the clinical procedure.

Referring now to FIGS. 5A and 5B, a perspective view of a pullback module operably attached to a portion of an imaging probe and a delivery catheter, and an exploded view of a portion of a pullback module are illustrated, respectively, consistent with the present inventive concepts. In FIG. 5A, pullback connector 180 is positioned operably attached to carrier 855 of pullback module 880. Hub 861 is positioned within retention port 884. Connector 82 is positioned distal to projection 862, onto which connector 82 can be slidingly received to form a fluid tight connection between delivery catheter 80 and adapter 860.

In FIG. 5B, a two-piece embodiment of carrier 855 is illustrated in an exploded view. Carrier 855 can include an inner portion, portion 8551, which is slidingly positioned within an outer portion, portion 8555. Inner portion 8551 can comprise one or more alignment features, tracks 8552a,b shown. Outer portion 8555 can comprise one or more alignment features, rails 8556a,b (rail 8556b shown), that mate with tracks 8552a,b of inner portion 8551. Inner portion 8551 can comprise one or more operator manipulatable projections, projections 8553a,b shown. Each projection 8553 can comprise an engagement portion, teeth 8554, such as teeth 8554a shown. Teeth 8554 can be positioned on the outward-facing surface of projections 8553, and can be aligned with one or more mating engagement portions of outer portion 8555, teeth 8557a,b shown. Inner portion 8551 can be constructed and arranged to be slidingly positioned within outer portion 8555 in a releasably locking manner, such that teeth 8554 can engage with teeth 8557 to maintain the longitudinal position between portions 8551 and 8555. In some embodiments, projections 8553 are constructed and arranged to be elastically deformed inward when grasped and pinched (e.g. pinched together) by an operator of system 10. When grasped in such a manner, teeth 8554 and 8557 disengage, allowing the operator to adjust the longitudinal position of inner portion 8551 relative to outer portion 8555. In some embodiments, with connector 180 positioned within carrier 855 while carrier 855 is in its fully advanced position, and imaging probe 100 is advanced to a desired location, an operator can retract inner portion 8551 to fully engage with connector 180. For example, due to this adjustment, when carrier 855 is retracted during the pullback procedure there is no initial gap between the distal wall of inner portion 8551 and the distal edge of connector 180. The position of inner portion 8551 can be configured to allow position adjustment up to a maximum level, such as a maximum of at least 5 mm, such as at least 7 mm, such as up to 15 mm. Adjustment of inner portion 8551 can be performed in increments as small as 1.0 mm, 0.7 mm, 0.5 mm, and/or 0.3 mm.

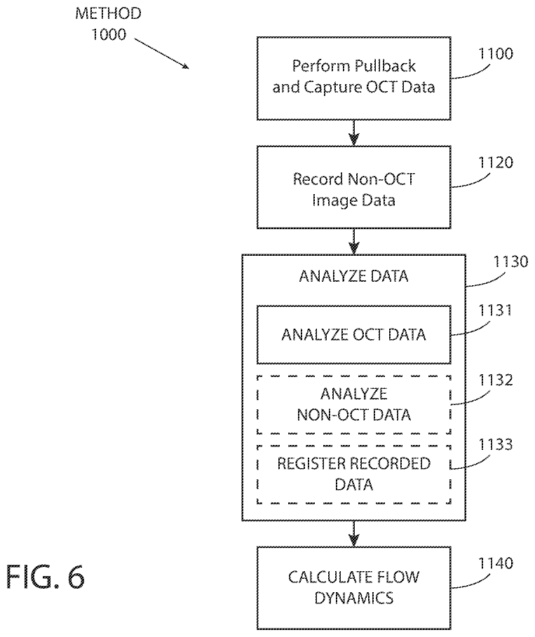

Referring now to FIG. 6, a flowchart of a method of calculating blood flow parameters within a vessel is illustrated, consistent with the present inventive concepts. Method 1000 of FIG. 6 is described using system 10 described in reference to FIGS. 1-3. The method of FIG. 6 is described where system 10 is used to create image data from one or more arteries of the patient's heart. It should be considered within the spirit and scope of this application that a similar method can be applied to other blood vessels and/or other internal locations of the patient's anatomy (e.g. arteries and/or veins of the patient's brain or peripheral vasculature).

In Step 1100 imaging probe 100 is inserted into a selected vessel of a patient, a pullback is performed within the selected vessel, and OCT Data is recorded, as described herein. In some embodiments, the selected vessel comprises one or more arteries, veins, and/or other conduits (“artery” or “arteries” herein). The selected vessel may have been previously selected for diagnosis by an operator of system 10 (e.g. a clinician of the patient), such as an artery which may require interventional treatment. Alternatively or additionally, the operator of system 10 may select a vessel for diagnosis during Step 1100. In some embodiments, the selected vessel is manually input into system 10, such as by the operator of system 10. In some embodiments, the selected vessel comprises an artery selected from the group consisting of: left circumflex (LCx); right coronary artery (RCA); left anterior descending (LAD); and combinations of these. In some embodiments, data is input into system 10 by an operator during a processing step, such as Step 1130 described herein. For example, while system 10 (e.g. algorithm 51) performs one or more background calculations, system 10 can be further configured to accept operator input such that an overall procedure time is reduced (e.g. while system 10 is processing data, the operator performs required and/or other data entry).

Imaging probe 100 is inserted into the selected vessel (referred to herein as the “selected vessel”, the “imaged vessel”, the “selected artery”, the “imaged artery”, the “selected vein”, or the “imaged vein”, as appropriate). When two or more arteries are selected for diagnosis, Step 1100 and the following steps can be repeated for each selected artery. Probe 100 placement (e.g. positioning of optical assembly 115) is performed to effectively obtain the images required for one or more various calculations to be performed by system 10 (e.g. flow calculations). The anatomical location at which the pullback is to begin (e.g. the position of optical assembly 115 at the start of a pullback) is selected as a location beyond (i.e. distal to) the distal end of the distal-most diseased portion of the artery (e.g. the distal-most portion of a lesion). The location at which the pullback is to end (e.g. the position of optical assembly 115 at the end of a pullback) is selected as a location within the most distally placed guide catheter through which imaging probe 100 is inserted (e.g. a distal portion of a delivery catheter 80).

In some embodiments, the operator can enter one, two, or more patient parameters into system 10, such as via user interface 55 of console 50. The one, two, or more patient parameters entered can be selected from the group consisting of: patient weight; existence of one or more patient diseases (e.g. in addition to cardiovascular disease such as diabetes if applicable); gender; age; height; TIMI score; previous coronary interventions such as stent implants, bypass grafts, and the like; and combinations of these.

In Step 1120, system 10 can be used to capture non-OCT imaging data of the selected patient, such as via second imaging device 15 described herein. As used herein, “non-OCT data” shall include but not be limited to: angiography image data; ultrasound image data; MRI image data; PET Scan image data; and/or other non-OCT imaging data. In some embodiments, non-OCT data comprises two or more of: angiography image data; ultrasound image data; MRI image data; PET Scan image data; and/or other non-OCT imaging data. In some embodiments, non-OCT data comprises angiography image data, and one or more of: ultrasound image data; MRI image data; PET Scan image data; and/or other non-OCT imaging data.

This non-OCT data can be stored in memory of system 10, such as memory of processor 52 of console 50. For example, angiography images of one or more vessels can be obtained using contrast injections, wherein the angiography data is saved in system 10 memory (e.g. capturing and/or saving of data performed automatically by system 10). Alternatively or additionally, the operator may manually enter relevant patient data (e.g. data similar to and/or extracted from angiography data or other non-OCT data) into console 50 (e.g. via a keyboard or other user input component 57 of user interface 55). Step 1120 can be performed (e.g. repeated, in whole or in part) during any of the steps of the method of FIG. 6 described herein. For example, Step 1120 can be performed prior to, during, and/or after the pullback performed in Step 1100 described herein.

In Step 1130, the OCT recorded data is analyzed. In some embodiments, both OCT data and non-OCT data (e.g. angiography data) is analyzed (e.g. OCT data is analyzed in combination with non-OCT data). Steps 1131, 1132, and/or 1133 (or portions of these) can be performed sequentially, simultaneously, and/or they can be interleaved as the recorded data is analyzed by system 10.

In Step 1131, recorded OCT data is analyzed by system 10. In some embodiments, the OCT data can be analyzed to identify one or more of the following: lumen boundaries; side branches; healthy (e.g. non-diseased) portions of lumen; diseased portions of lumen; type of disease imaged; the location of a guidewire within the image; and combinations of one or more of these. In some embodiments, the guidewire is removed from the OCT data. In some embodiments, system 10 identifies healthy sections of the imaged artery based on the OCT data. Healthy sections can be determined by identifying visible intima, media, and/or adventitia layers within the OCT data. In some embodiments, the myocardial mass can be estimated based on the diameter of one or more identified healthy sections of the imaged artery. In some embodiments, the estimate of the myocardial mass can be based on both OCT data and non-OCT data (e.g. angiography data).

System 10 can comprise a weighting function (e.g. where algorithm 51 comprises the weighting function), where the weighting function is configured to prioritize data (e.g. prioritize a data type) in one or more calculations, for example to preferentially bias a calculation based on OCT data vs non-OCT data, or vice versa. In some embodiments, system 10 (e.g. via algorithm 51) identifies the presence of disease proximate one or more side branches of an imaged artery. For example, if disease is detected proximate a side branch (e.g. disease is detected inside the side branch), the weighting function can be configured to prioritize angiography data related to the diseased side branch (e.g. to preferentially bias a calculation toward weighting angiography above OCT data).

System 10 can be configured to calculate (e.g. via algorithm 51) the branch angle of a side branch from the imaged artery. In some embodiments, the branch angle is used by algorithm 51 to calculate the side branch vessel diameter. System 10 can be configured to reconstruct at least a portion of the side branch from the OCT data (e.g. from the image slices of the OCT data), and/or from non-OCT data (e.g. from angiography data). In some embodiments, system 10 is configured to calculate the relationship between the side branch angle and the diameter of the side branch (e.g. the size of the side branch relative to the size of the imaged artery). In these embodiments, if the relationship between branch angle and branch diameter is outside of an expected range, system 10 can be configured to “flag” this anomaly (e.g. identify the anomaly and store the associated information), and/or to alert the operator of system 10 of this anomaly.

In some embodiments, system 10 is configured to identify a portion of OCT data representing the minimal lumen diameter (e.g. a portion of healthy tissue with the minimal lumen diameter). Additionally, system 10 can be configured to identify between two and five sections of the OCT data along the length of the lumen imaged during the pullback (e.g. equally spaced sections along the length of the lumen imaged). The identified sections can be used to estimate the non-diseased vessel size (e.g. lumen diameter) of the selected artery. In some embodiments, at least one of the identified sections comprises a proximal section, and at least one of the sections comprises a distal section (e.g. proximal and distal sections proximate the proximal and distal ends, respectively, of the lumen imaged). In some embodiments, the proximal and/or distal sections are within the proximal and distal 10%, respectively, of the length of the lumen imaged. In some embodiments, a weighting function is configured to apply a weight (i.e. a weighting factor) to each section based on a confidence level of the OCT data for that section. Confidence can be determined in several ways, for example the proportion of unambiguous lumen detected in a single frame (for example, >75% of the circumference should be unambiguous for high confidence), and/or the amount of change in lumen area from frame to frame (e.g. discontinuous frames should be given less weight). Confidence is also obtained from the deviation of circularity of the cross section of the imaged vessel.

In some embodiments, system 10 (e.g. via algorithm 51) identifies areas of disease based on OCT data and/or non-OCT data. In some embodiments, types of disease can be identified by system 10, such as when system 10 identifies plaque composition of a diseased segment of an artery.

In some embodiments, system 10 (e.g. via algorithm 51) determines the time required for contrast or other flush media (e.g. injectate 21) to clear from the selected vessel segment (e.g. segment to be imaged) after injector 20 has stopped an injection into the selected vessel (otherwise known and used herein as the “persist time”). In other words, the persist time is the length of time between the cessation of flush media delivery and the time point at which sufficient blood has returned to the vessel segment being imaged to prevent acceptable images of that vessel segment to be obtained (e.g. imaging of the luminal wall is obscured by the returning blood). The persist time is indicative of the relative blood flow in the chosen vessel segment being imaged. Persist time can be measured with a rotating, but longitudinally stable core 110 (e.g. core 110 is not being pulled back nor advanced). The persist time can be calculated based on OCT data and/or non-OCT data (e.g. angiography data). Algorithm 51 can receive a signal (e.g. a signal from injector 20) indicating the injection status of injector 20 (e.g. indicating when an injection starts and/or stops). Additionally or alternatively, a functional element of system 10 (e.g. functional element 89 of delivery catheter 80) can comprise a pressure transducer configured to monitor the fluid pressure within delivery catheter 80. The pressure within delivery catheter 80, as provided via a signal from the pressure-sensor-based functional element 89, can be analyzed by algorithm 51 to determine the status of a flushing procedure (e.g. status related to initiation and/or cessation of injection of flushing media).

In Step 1132, non-OCT data (e.g. angiography data) can be analyzed. In some embodiments, non-OCT data is analyzed to identify one or more of the following: vessel geometries (e.g. curves, tapers, and/or trajectories); side branch locations (e.g. the size and position of side branches of one or more main arteries to be diagnosed); vessel lengths; vessel diameters; and combinations of one or more of these. In some embodiments, the non-OCT data can be analyzed to estimate one or more of the following: myocardial mass; collateral flow; size of the heart area (myocardium) being supplied by an artery (e.g. a selected artery); and combinations of one or more of these. In some embodiments, the non-OCT data analyzed in Step 1132 comprises angiography image data and one or more of: ultrasound image data; MM image data; PET Scan image data; and/or other non-OCT imaging data. In some embodiments, non-OCT data comprises angiography data which is converted by system 10 into QCA data. In some embodiments, non-OCT data comprises PET-scan data that system 10 converts into myocardial damage data (e.g. where system 10 adjusts vessel size in a damaged area).

In Step 1133, OCT data and non-OCT data (e.g. angiography data) can be registered (e.g. correlated). In some embodiments, the data can be registered using the location, size, and shape of one or more side branches of the selected artery. In some embodiments, system 10 can comprise a digital model of the expected branching of one or more of the major vessels of the heart, for example the LCx, RCA, and/or LAD arteries. In some embodiments, the digital model is used by system 10 to register the data.

In Step 1140, values of cardiovascular flow dynamic parameters (e.g. flow rate, fluid pressure, pressure drop over a distance, fractional flow reserve, and the like) can be calculated based on the analyzed data (e.g. the OCT data and/or Non-OCT data collected and/or analyzed). In some embodiments, system 10 is configured to estimate microvascular resistance distal to the selected artery.

In some embodiments, the measured size of the imaged artery is adjusted by system 10 (e.g. the size of the imaged artery as determined in step 1130 is adjusted by algorithm 51). System 10 can adjust the measured size of the imaged artery based on Murray's Law. For example, system 10 can assume constant sheer stress in all non-diseased areas of the imaged artery. This adjustment can minimize the error function of flow calculated by system 10 using Murray's Law. In some embodiments, flow calculations are adjusted using a weighting function that is based on the confidence of the analyzed data. For example, if Murray's law suggests an adjustment to the measured size and the confidence of the imaged cross section is relatively low then system 10 can be configured to adjust the flow calculations based on the Murray's law suggested adjustments. Alternatively, if the confidence is high, one or more adjustments based on Murray's law can be ignored by system 10.

The size of the myocardium being supplied can be estimated by system 10 (e.g. using algorithm 51) based on the selected artery vessel type and/or calculated size. In some embodiments, the estimated myocardial size can be compared to the estimated size calculated based on non-OCT data (e.g. angiography data), such as described in Step 1132. In some embodiments, if the non-OCT-based estimate and the OCT-based estimate vary, the operator is alerted by system 10 (e.g. via a user display 56 or other user output component of interface 55). In some embodiments, system 10 accepts user input to adjust the estimate (e.g. via user input 57 of user interface 55). In some embodiments, the OCT-based estimate is given a higher weight (e.g. by a weighting function of system 10) than the angiography-based estimate (e.g. the OCT-based estimate is favored by system 10). In some embodiments, the data is displayed to the operator as a color map (e.g. a color map shown in display 56). In some embodiments, the color map can indicate the estimated myocardial size being supplied by a selected artery (e.g. the amount of myocardial tissue supplied by a selected artery). In some embodiments, hyperemic microvascular resistance is estimated by system 10 (e.g. via algorithm 51) for all or at least a portion of the heart outside the imaging area.

In some embodiments, the pressure throughout the vasculature of the heart (the “coronary tree”) is calculated. In some embodiments, fractional flow reserve (FFR) is calculated (e.g. calculated from the lowest pressure). The FFR can be calculated for a selected artery, such as for each selected artery (e.g. each artery selected and imaged using OCT). In some embodiments, system 10 can be configured to alert the operator (via user interface 55) if one or more of the following is detected: significant collateral flow is found (e.g. flow above a system 10 threshold); a myocardial estimate varies significantly (e.g. the variance is above a system 10 threshold) between an OCT-based estimate and a non-OCT-based estimate (e.g. angiography-based estimate); a TIMI score indicates compromised myocardial tissue; a region of no-flow is detected angiographically (e.g. indicating a blockage); and combinations of one or more of these.

In some embodiments, system 10 (e.g. via algorithm 51) can calculate one or more cardiovascular flow dynamic parameters based on the persist time, as described herein, of contrast and/or other injectate 21 within the selected vessel segment.

In some embodiments, persist time can be used as an estimate of local blood flow (e.g. similar to TIMI flow scores used in angiography). Estimates of local blood flow (e.g. as determined by system 10) can be used by system 10 to improve the accuracy of calculations of cardiovascular flow dynamics.

In some embodiments, OCT data and non-OCT data (e.g. angiography data) is displayed (e.g. on display 56), for example in an overlay arrangement (e.g. OCT data is overlaid on angiography data and/or other non-OCT data), such as after the multiple sets of data have been registered.

In some embodiments, a graph of lumen diameter is displayed, such as a graph of lumen diameter along the length of a pullback (also referred to herein as a “lumenogram”). In some embodiments, the lumenogram is based on an average diameter (the average of multiple chords drawn through the lumen centroid), or it can be based on an “effective” diameter, such as the diameter of a circle which has the same area as the area of the irregular lumen itself. The effective diameter can be determined (e.g. by an algorithm of system 10) using Green's theorem:

½ [symbol] xdy−ydx

For image data captured by imaging probe 100, once a lumen boundary has been identified (e.g. by an algorithm of system 10), use of Green's theorem is a fast and simple way to calculate the area of an irregular object (e.g. a lumen whose periphery is not a perfect circle). However, in some applications of system 10, a lumenogram based on the effective diameter is not the most appropriate way to provide physiologic information regarding a vessel segment (e.g. pressure drop along the vessel segment). In some embodiments, system 10 calculates a “hydraulic diameter” for various locations along a lumen segment, such as a hydraulic diameter determined by an algorithm of system 10 that is based on the D'Arcy-Weisbach Equation—a modified form of which is:

$$Δ p = λ ( \frac{l}{{d}_{h}} ) ( \frac{p {v}^{2}}{2} )$$

where

Δp=major friction pressure loss (Pa, (N/m²), lb/ft²);

λ=friction coefficient;

l=length of vessel segment (m, ft);

dₕ=hydraulic diameter (m, ft);

p=density of fluid (kg/m³, slugs/ft³); and

v=flow velocity (m/s, ft/s)

It can be seen that a pressure drop in a vessel segment depends more directly on the hydraulic diameter, not the area or effective diameter. The hydraulic diameter is defined as:

$${d}_{h} = \frac{4 A}{P}$$

where P is the perimeter of lumen; and

A is the area

In order to calculate the hydraulic diameter, an algorithm of system 10 (e.g. algorithm 51) can calculate the perimeter of multiple axial locations along the vessel segment. Note that the hydraulic diameter and the effective diameter are the same for a perfect circle. The less circular the cross section, the more the hydraulic diameter differs from the effective diameter, and the less accurate an effective diameter-based lumenogram becomes for providing pressure drop information.

TABLE 1

| | | Circle | | Circle or | |
|---|---|---|---|---|---|
| Ellipse | | Effective | Average | Ellipse | Hydraulic |
| A-Axis | B-Axis | Diameter | Diameter | Area | Diameter |
| 2.0 | 50.0 | 10 | 34.0 | 78.54 | 3.14 |
| 4.0 | 25.0 | 10 | 18.0 | 78.54 | 6.07 |
| 6.0 | 16.7 | 10 | 13.1 | 78.54 | 8.35 |
| 8.0 | 12.5 | 10 | 11.0 | 78.54 | 9.64 |
| 10.0 | 10.0 | 10 | 10.0 | 78.54 | 10.00 |
| 12.0 | 8.3 | 10 | 10.8 | 78.54 | 9.76 |
| 14.0 | 7.1 | 10 | 11.7 | 78.54 | 9.22 |
| 16.0 | 6.3 | 10 | 12.8 | 78.54 | 8.57 |
| 18.0 | 5.6 | 10 | 13.9 | 78.54 | 7.93 |
| 20.0 | 5.0 | 10 | 15.0 | 78.54 | 7.33 |

As shown in Table 1, it can be seen that even though as an ellipse changes shape over a factor of ten, the effective diameter and area are invariant. However, use of the hydraulic diameter in a lumenogram would more accurately indicate that the expected pressure drop varies considerably and in the correct sense (i.e. a “squashed” ellipse has a higher pressure drop, as would be expected).

System 10 can determine the perimeter of various (e.g. relatively continuous) locations along a vessel segment using the following calculation:

Perimeter= [symbol] ds

where ds is the infinitesimal length segment around the closed perimeter.

In turn, ds is given by:

$$ds = \sqrt{1 + {( \frac{d y}{d x} )}^{2} d x}$$

In some embodiments, system 10 displays a lumenogram based on the hydraulic diameter for visually conveying pressure drops along a vessel, and it displays a lumenogram based on the effective diameter for visually conveying information related to sizing and/or otherwise selecting a stent for placement in the vessel being imaged. Alternatively or additionally, specific hydraulic diameter and/or effective diameter information can be displayed by system 10 (e.g. in a popup window including numeric information) at one or more locations along a displayed lumenogram.

In some embodiments, system 10 displays a lumenogram based on hydraulic diameter along one axis, while the longitudinal position of the pullback image is displayed on the other axis. In some embodiments, displaying of numeric value of the hydraulic diameter can be avoided as this could be misconstrued by the operator (e.g. the hydraulic diameter is displayed as a visual indicator only). Regions where the hydraulic diameter is changing quickly can be highlighted and/or color coded by system 10. In some embodiments, the hydraulic axis is displayed as a simple line plot. Alternatively or additionally, the hydraulic axis can be displayed to resemble an angiogram (e.g. a shadow-gram). This form of display is more effective at conveying the pressure drops in a vessel segment, which is the primary factor in deciding whether and how to treat a vessel.

System 10 can provide a lumenogram that is based on hydraulic diameter to provide pressure drop information, while providing numeric information (typically effective diameter and/or area) that are not linear-scaled to the display (e.g. not including a diameter and/or area display axis that is linearly proportional to the numerically displayed value).

In some embodiments, one or more side branches (or portions thereof) of the selected artery are displayed. In some embodiments, the operator can add data (e.g. manually add data) to system 10 indicating that disease is present proximate the displayed OCT data, for example at the proximal or distal end of the lumen, or within a side branch (e.g. the operator visually determines that disease is present via information provided by second imaging device 15 and manually enters associated disease type and/or disease location data into system 10). In some embodiments, calculated vessel sizes are displayed along with the OCT images and/or non-OCT images (e.g. angiography images). In some embodiments, system 10 allows the operator to edit the displayed calculated values (e.g. in order to allow an operator-initiated manual adjustment of a system 10 calculated value).

In some embodiments, an operator of system 10 plans a treatment procedure based on the displayed data. In some embodiments, the operator indicates a length and location of a “treatment area” to be evaluated by system 10 (e.g. the operator indicates the treatment area by clicking on the displayed image). In some embodiments, system 10 displays the estimated vessel diameter at the proximal and distal ends of the selected treatment area. In some embodiments, the vessel diameters are estimated by system 10 (e.g. via algorithm 51) using Green's Theorem. In some embodiments, system 10 displays one or more pieces of information related to the selected treatment area, for example any warnings associated with the treatment area and/or the plaque composition within the treatment area. In some embodiments, post-treatment flow dynamics (e.g. FFR) can be estimated assuming the planned treatment (e.g. stenting) opens the treated vessel to the estimated diameter at the proximal and distal ends of the selected treatment area (e.g. where two diameters can be used to indicate vessel tapering). In some embodiments, the post-treatment flow dynamics can be estimated based on the calculated pre-treatment flow dynamics and the proposed treatment. In some embodiments, the operator can vary the proposed treatment area, and system 10 can update the flow dynamics based on the new treatment area. In some embodiments, the operator can indicate two or more treatment areas (e.g. two or more non-contiguous treatment areas), and post-treatment flow dynamics can be estimated for each treatment area.

As described herein, system 10 can be configured to calculate the flow characteristics through one or more blood vessels that have been imaged using probe 100 (e.g. via one or more pullbacks). System 10 can be configured to calculate the flow dynamics (e.g. flow field and/or pressure drop) through an imaged vessel (e.g. an artery to which a treatment has been or will be performed). In some embodiments, system 10 calculates the flow and pressure drop of coronary vessels using a full 3D Navier-Stokes simulation of the vessel imaged using imaging probe 100. In some embodiments, system 10 is configured to include in its analysis (e.g. via algorithm 51) numerous geometric and other features of the imaged area, such as when every significant morphological feature is represented. System 10 can directly measure the pressure in the vessel, and the FFR at the locations along the length of the vessel may be calculated, as described herein. Regions where the rapid changes in FFR occur (e.g. as identified by system 10) can be used to justify intervention (e.g. stenting). In some embodiments, system 10 calculates flow dynamics (e.g. flow dynamics of an imaged cardiac vessel and/or other vessel) based on hydraulic diameter data (e.g. hydraulic diameter data that is calculated by system 10 based on the image data collected by system 10). Alternatively or additionally, system 10 calculates flow dynamics based on average diameter data calculated by system 10 based on the image data collected by system 10. In some embodiments, system 10 calculates flow dynamics based on both average diameter and hydraulic diameter information (e.g. as collected by system 10).

Referring now to FIG. 7, a flow chart of a method of performing a pullback procedure based on a pullback triggering condition is illustrated, consistent with the present inventive concepts. Method 4000 of FIG. 7 is described using system 10, such as is described in reference to FIGS. 1-3 herein. In Step 4010, optical assembly 115 is positioned (e.g. by a clinician) distal to a location to be imaged by system 10. The imaging location can comprise a segment of a luminal vessel, such as a blood vessel. Once optical assembly 115 is positioned distal to the imaging location, system 10 is in a condition for performing a pullback procedure for that location, and method 4000 continues to Step 4020.

In Step 4020, system 10 enters a “ready” for pullback condition and begins monitoring for a “pullback triggering condition”, such that the trigger can cause the pullback to start immediately. In some embodiments, algorithm 51 analyzes information gathered from one or more components of system 10, such as information recorded by imaging assembly 300, second imaging device 15, patient monitoring device 17, functional element 199, and/or functional element 89. Algorithm 51 can determine, based on the analysis of the information, if a pullback triggering condition for initiating the pullback procedure has occurred. In some embodiments, the pullback triggering condition is based on a point in the patient's heart cycle, for example, when patient monitoring device 17 comprises an electrocardiogram (EKG) monitor. Algorithm 51 can be configured to trigger the pullback during a particular part of the heart cycle, for example, a part of the heart cycle in which there is relatively low motion of the heart muscle proximate the imaging location (e.g. a resting portion of the heart cycle). In some embodiments, algorithm 51 is configured to trigger the pullback when the T-Wave of the EKG is detected. In some embodiments, the pullback triggering condition is based on angiographic data, for example, when second imaging device 15 comprises a device configured to perform angiography such as rotational angiography. In some embodiments, algorithm 51 analyzes the motion of the heart (e.g. the motion of the heart muscle) based on the angiographic data. Additionally or alternatively, algorithm 51 can analyze angiographic data to determine a point of the heart cycle. System 10 can be configured to trigger the pullback when the motion of the heart is determined to be minimal based on this analysis by algorithm 51. In some embodiments, the position of a marker 131 of imaging probe 100 is analyzed by algorithm 51 (e.g. by analyzing angiographic data that includes a reflection of a radiopaque-based marker 131) and the pullback is triggered when the marker 131 is determined to be stationary or at least its movement is minimal (e.g. the pullback is triggered when the movement of the marker 131 is determined to be at a relative minimum as compared to its movement at other points of the heart cycle). Alternatively or additionally, the position of distal tip 119 can be analyzed by algorithm 51 to determine a time of minimal motion of the vessel segment to be imaged.

In some embodiments, algorithm 51 is configured to analyze data (e.g. OCT data) to detect an EKG pattern. For example, algorithm 51 can analyze OCT data comprising image motion and/or speckle analysis data to detect an EKG pattern. In some embodiments, a continuous EKG pattern can be detected by algorithm 51 after a period of time (e.g. seconds, minutes). The generated EKG pattern can be used to predict the timing of one, two, or more future heart beats, such as one or more future heartbeats whose timing is used to trigger a pullback procedure (e.g. a pullback procedure performed following the initiation of a flushing procedure). These embodiments allow for the detection of an EKG pattern (e.g. and an EKG-triggered pullback) that is based solely on OCT data, thereby eliminating the need for a separate device to provide an EKG signal (e.g. avoiding the need to connect to external triggers and/or precise synchronization with an angiogram).

In Step 4030, if a pullback triggering condition is detected, Method 4000 continues to Step 4040. If a pullback triggering condition is not detected, the operator of system 10 can cancel the automatic pullback detection in Step 4035. If the automatic pullback detection is not canceled in Step 4035, Method 4000 returns to Step 4030. If the automatic pullback detection is canceled in Step 4035, Method 4000 continues to Step 4050. In Step 4050, the pullback can be disabled and algorithm 51 stops monitoring for a pullback triggering condition. In some embodiments, after Step 4050, the operator can reenable a pullback and restart the monitoring (e.g. return to Step 4020). In Step 4030, if a pullback triggering condition is detected, Method 4000 continues to Step 4040, and a pullback is triggered.

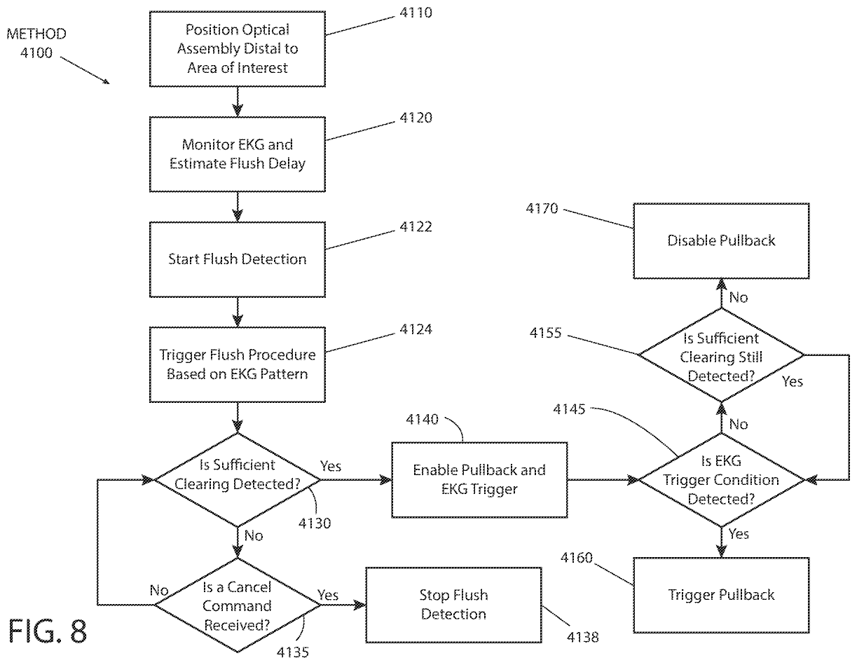

Referring now to FIG. 8, a flow chart of performing a pullback procedure based on multiple pullback triggering conditions is illustrated, consistent with the present inventive concepts. Method 4100 of FIG. 8 is described using system 10, such as is described in reference to FIGS. 1-3 herein. Method 4100 herein describes an embodiment wherein the first of multiple pullback triggering conditions comprises the detection of vessel segment clearing. For example, algorithm 51 can analyze data to determine that the vessel segment selected to be imaged is sufficiently cleared of blood for “proper” OCT imaging. In some embodiments, algorithm 51 can detect the vessel lumen based on one or more known lumen characteristics (e.g. pre-determined lumen characteristics). In some embodiments, the vessel lumen is detected using numeric characterization techniques (e.g. edge detection techniques), and/or using a trained neural network. Once a reliable lumen is detected (e.g. continuously detected over several contiguous frames, such as between two and ten contiguous frames) then adequate vessel clearing has been established and pullback can commence. The second of the multiple pullback triggering conditions comprises detection of a point in the patient's heart cycle (e.g. pullback is triggered during an optimal point of the heart cycle for OCT imaging, such as when algorithm 51 analyzes an EKG signal to detect a heart cycle point with minimal motion). Steps 4120, 4122, and 4124 shown describe an embodiment in which a flush is started based on a “flush trigger condition”, such as when a point in a patient's heart cycle is reached. In some embodiments, a flush is started at the operator's discretion (e.g. an action by the clinician, not triggered by system 10).

In Step 4110, optical assembly 115 is positioned distal to a location to be imaged by system 10. The imaging location can comprise a segment of a luminal vessel, such as a blood vessel. After optical assembly 115 is positioned distal to the imaging location, system 10 is in a ready condition for performing a pullback procedure for that location, and Method 4100 continues to Step 4120. In some embodiments, the first of the multiple pullback triggering conditions detected by algorithm 51 in Method 4100 comprises detection of vessel segment clearing (e.g. algorithm 51 detects that the vessel segment selected to be imaged is sufficiently cleared of blood for proper OCT imaging).

In Step 4120, algorithm 51 monitors the patient EKG signal, and estimates the delay time between the time an injection of flush is initiated and the time when the selected vessel segment will be sufficiently cleared of blood for proper OCT imaging. In some embodiments, algorithm 51 predicts the optimal point in the heart cycle (e.g. based on the EKG signal and the estimated delay time) to trigger (e.g. begin) an injection of flush such that the selected vessel segment is sufficiently cleared during an optimal portion of the heart cycle for proper OCT imaging (e.g. an optimal point of the heart cycle for the pullback to begin). In some embodiments, the estimated delay time is based on the anatomic position of the selected vessel and/or the length of delivery catheter 80 (e.g. the catheter through which flush is injected into the selected vessel).

In Step 4122, algorithm 51 can initiate flush detection (e.g. upon a detection of sufficient clearing of the selected vessel segment for proper OCT imaging). In some embodiments, algorithm 51 is configured to detect sufficient clearing as described herein in reference to FIG. 8. After flush detection is initiated, in Step 4124, algorithm 51 monitors the patient EKG signal and begins a flush procedure based on the predictions produced in Step 4120.

In Step 4130, if sufficient vessel segment clearing is not detected, the operator of system 10 can cancel the pullback in Step 4135, and in Step 4138, the flush detection is stopped. In some embodiments, if sufficient vessel segment clearing is not detected within a time period (e.g. a period of time from the start of the flush procedure), algorithm 51 can enter an alert mode and the pullback can be canceled automatically by system 10. In some embodiments, the time period can be no more than 5 seconds, such as no more than 2 seconds since the start of the flush procedure. If sufficient vessel segment clearing is detected, Method 4100 continues to Step 4140.

In Step 4140, after sufficient vessel segment clearing has been detected, algorithm 51 enables the automatic pullback of imaging probe 100 and begins monitoring the patient EKG signal for a pullback triggering condition based on the patient's heart cycle. In some embodiments, algorithm 51 is configured to trigger the pullback when the T-Wave is detected in the EKG signal. In Steps 4145 and 4155, algorithm 51 monitors for the heart cycle-based pullback triggering condition while monitoring if sufficient vessel segment clearing is still present (e.g. still detected). In Step 4155, if sufficient vessel segment clearing is not detected (e.g. no longer detected), Step 4170 is performed in which algorithm 51 enters an alert mode and disables the pullback. In Step 4145, if the EKG pullback triggering condition is detected, Method 4100 continues to Step 4160, and the pullback is triggered.

While the embodiments of FIG. 8 have been described as including a first pullback trigger related to vessel segment clearing, and a second pullback trigger related to the patient's heart cycle, alternative and/or additional patient, system, and/or method parameters can be monitored by system 10 (e.g. and analyzed by algorithm 51) and used to trigger a pullback.

Referring now to FIG. 9, an OCT image showing a vessel suspended in cerebral fluid is illustrated, consistent with the present inventive concepts. The image shown in FIG. 9 can be displayed to an operator (e.g. on display 56 of system 10 described herein). In some embodiments, system 10 is configured such that optical assembly 115 of imaging probe 100 is positioned within a first location (e.g. a blood vessel), in order to capture images of locations outside of the first location (e.g. outside of the blood vessel or other vessel of the patient as described herein). For example, optical assembly 115 can be positioned in a first blood vessel (e.g. an artery or a vein) of the patient's heart, such as to provide an image of a location proximate but outside of the heart's first blood vessel walls (e.g. a location in which flush media is also delivered), such as to provide an image of a second blood vessel of the heart, a chamber of the heart, heart wall tissue, and/or any heart tissue. Optical assembly 115 can be positioned in a first blood vessel (e.g. an artery or a vein) of the patient's brain, such as to provide an image of a location proximate but outside of the brain's first blood vessel walls (e.g. via delivered and/or reflected light of probe 100 that passes through cerebral spinal fluid of the brain), such as to provide an image of a second blood vessel of the brain, a ventricle of the brain, cortical tissue of the brain, white matter of the brain, grey matter of the brain, the fornix, the hippocampus, and/or any brain tissue (e.g. healthy and/or diseased tissue as described herein). Optical assembly 115 can be positioned in a first vessel (e.g. an artery, a vein, or a channel of the spinal cord such as the epidural space, subdural space, or subarachnoid space) proximate the patient's spine, such as to provide an image of a location proximate but outside of the spine's first vessel walls, such as to provide an image of a second vessel of the spine, a different channel of the spinal cord, nerve bundles of the spinal cord, and/or any spine tissue. Optical assembly 115 can be positioned in a first vessel (e.g. an artery, a vein, channel, chamber, or any vessel of the patient) that is proximate an anatomical location selected from the group consisting of: a perivascular structure or other perivascular location of the patient; a subarachnoid space of the patient; an arachnoid trabeculae of the patient; and combinations of these, such as to provide an image within the vessel, and/or outside of the vessel in which optical assembly 115 is positioned. Optical assembly 115 can be positioned in a first vessel (e.g. an artery, a vein, channel, canal, duct, chamber, or any vessel of the patient) at a location that is proximate target tissue or other target location of the patient to be diagnosed and/or treated (e.g. a target location comprising tumor tissue, neuritic plaque, amyloid plaque, a cerebral infarct, atherosclerosis, and/or other diseased and/or non-diseased tissue outside of the first vessel), such as to provide an image of the target tissue, where the image provides diagnostic information of the target tissue and/or is used to perform and/or assess a treatment of the target tissue. Optical assembly 115 can be positioned within a lumen of intracranial vasculature to produce an image of one or more physiologic markers of vascular dementia and/or Alzheimer's disease. The physiologic markers can comprise markers within the vessel in which optical assembly 115 is placed, and/or outside (e.g. but proximate) the vessel in which optical assembly 115 is placed. The physiologic markers of vascular dementia and/or Alzheimer's disease can comprise one, two, or more markers selected from the group consisting of: amyloid plaques; neuritic plaques; cerebral infarcts; atherosclerosis; and combinations thereof. In some embodiments, algorithm 51 is configured to analyze OCT data to identify one, two or more physiologic markers of a disease (e.g. a vascular disease). In some embodiments, the one or more physiologic markers (e.g. markers of disease) identified by algorithm 51 comprise tumor tissue, amyloid plaques, neuritic plaques, cerebral infarcts, and/or atherosclerosis. System 10 can be configured to provide image data that comprises (e.g. identifies in a provided image) one or more physiologic markers (e.g. markers of disease), such as: tumor tissue, amyloid plaques, neuritic plaques, cerebral infarcts, and/or atherosclerosis. Delivery catheter 80 can be positioned within a body space (e.g. a non-luminal space) proximate target tissue to be imaged, and optical assembly 115 can be positioned within a portion of delivery catheter 80 (e.g. and retracted within catheter 80 during a pullback procedure). In some embodiments, system 10 is configured to provide an extended scan range for producing an image, such as a scan range of at least 7 mm (e.g. to provide an image with a 14 mm diameter), such as a scan range of at least 11 mm. In some embodiments, system 10 is configured to provide image data for locations at least 5 mm, at least 7 mm, or at least 9 mm outside of a vessel (e.g. a blood vessel or other patient vessel of at least 0.9 mm, at least 1.4 mm, or at least 2 mm in diameter). In some embodiments, optical assembly 115 comprises a focal length of at least 5 mm, such as a focal length of at least 7 mm, 10 mm, 12 mm, and/or 15 mm. In these embodiments, the working distance can comprise a distance of at least 10 mm, at least 15 mm, or at least 20 mm.

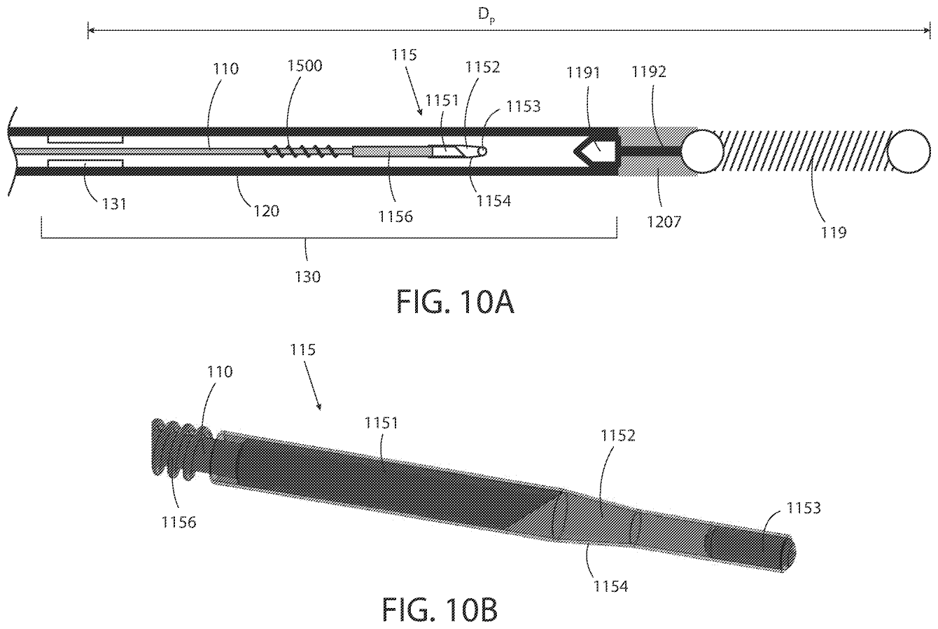

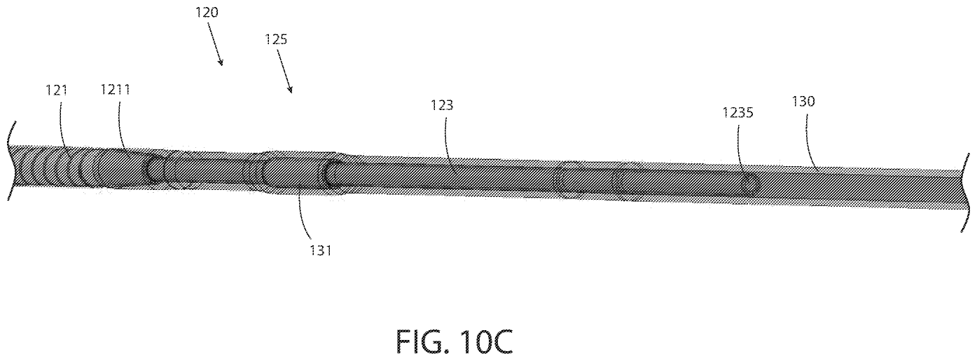

Referring now to FIGS. 10A-10C, various views of portions of an imaging probe are illustrated, consistent with the present inventive concepts. In FIG. 10A, the distal portion of imaging probe 100 is illustrated. Shaft 120 surrounds core 110 and optical assembly 115. Distal tip 119 comprises a spring tip extending from the distal end of shaft 120. In some embodiments, distal tip 119 comprises a spring made of palladium. In some embodiments, distal tip 119 comprises one or more anchoring elements, retainer 1191. Retainer 1191 shown extends proximally from spring tip 119 and is anchored within the distal end of shaft 120. Retainer 1191 can be fixedly attached to the coil of spring tip 119 via a wire, core wire 1192. In some embodiments, the proximal end of retainer 1191 comprises limited surface area that is perpendicular to the distal end of optical assembly 115 (e.g. the proximal end of retainer 1191 is pointed, as shown). The shape of the proximal end of retainer 1191 can be selected to minimize the amount of light that is reflected off of retainer 1191 and directed back to optical assembly 115. In some embodiments, distal portion 1208 of shaft 120 comprises a portion distal to window 130, tip portion 1207. Tip portion 1207 can be configured to surround core wire 1192. Tip portion 1207 can comprise a heat shrinkable material configured to have its diameter reduced (e.g. shrunk) around core wire 1192 after retainer 1191 has been affixed within the distal end of window 130. In some embodiments, tip portion 1207 comprises an outer diameter (e.g. post heat-shrinking outer diameter) approximately equal to the outer diameter of window 130 and/or equal to the outer diameter of the spring portion of spring tip 119 (e.g. such as to provide a smooth outer diameter of imaging probe 100 from tip 119 to window 130). In some embodiments, tip portion 1207 comprises an inner diameter (e.g. post heat shrinking inner diameter) that is approximately equal to the outer diameter of core wire 1192. In some embodiments, tip portion 1207 is sized to help ensure core wire 1192 is concentrically positioned about the longitudinal axis of imaging probe 100.

Optical assembly 115 can be positioned at the distal end of optical core 110. Optical assembly 115 can comprise a lens assembly, assembly 1151, which is optically and physically coupled to the distal end of optical core 110. Lens assembly 1151 can comprise a GRIN lens comprising a beveled distal end. The beveled distal end of lens assembly 1151 can comprise a total internally reflective surface. Optical assembly 115 can comprise lens marker 1156, an element that can be imaged by a separate imaging device (e.g. imaging device 15 of FIG. 1). Marker 1156 can comprise a radiopaque marker configured to be imaged using fluoroscopy or other X-ray based imaging device. In some embodiments, marker 1156 comprises a wire coil helically wrapped about a distal portion of optical core 110. In some embodiments, marker 1156 abuts the proximal end of lens assembly 1151 (e.g. marker 1156 is proximate the splice between lens assembly 1151 and optical core 110). In some embodiments, marker 1156 comprises an outer diameter approximately equal to the outer diameter of lens assembly 1151. In some embodiments marker 1156 is positioned in direct contact with optical core 110 (e.g. in direct contact with the glass surface of optical core 110). In some embodiments, marker 1156 comprises a pitch at least 1.5 times greater than the diameter of the wire of marker 1156, such that marker 1156 comprises a spacing between the coils of at least half of the width of the wire of marker 1156. This spacing can increase the flexibility of marker 1156, such as to prevent stress concentrations in optical core 110 (e.g. stress concentrations at the proximal and/or distal end of marker 1156 that may be at an undesirable high level if marker 1156 is too stiff). In some embodiments, marker 1156 is adhered to optical core 110 using an adhesive. In some embodiments, the spacing between coils of marker 1156 is sufficient to allow adhesive to wick into the coils of marker 1156, providing a uniform distribution of adhesive about marker 1156. In some embodiments, marker 1156 is adhered using an adhesive that is visible under UV light. In some embodiments, in manufacturing, UV light can be used to inspect marker 1156 to make sure adhesive has been properly applied and distributed about the marker 1156 (e.g. about the coils of marker 1156). In some embodiments, the adhesive used to adhere marker 1156 is also configured to provide support to the splice between optical core 110 and lens assembly 1151.

An elongate tube, tube 1154, can surround at least a distal portion of optical core 110, lens assembly 1151, and a sealing element, plug 1153. In some embodiments, tube 1154 surrounds at least a portion of marker 1156. Tube 1154 can comprise a heat shrink material. Tube 1154 can comprise PET. At least a portion of tube 1154 can be adhesively attached, or otherwise attached, to at least a portion of lens assembly 1151, optical core 110, and/or plug 1153. Plug 1153 can be configured to prevent and/or at least limit the egress of gel 118 into a cavity created between lens assembly 1151 and plug 1153, space 1152 shown. Space 1152 can be filled with air and/or one or more other fluids. The fluid within space 1152 can be configured to provide desired optical properties between lens assembly 1151 and the fluid (e.g. configured to provide a glass-air interface).

Imaging probe 100 can comprise a fluid propulsion element, FPE 1500, positioned on optical core 110, proximal to lens assembly 1151. FPE 1500 can be similar to FPE 1500 described in reference to FIG. 1 herein.

FIG. 10B shows a perspective view of optical assembly 115 and the distal portion of optical core 110. In some embodiments, plug 1153 comprises a smaller outer diameter than lens assembly 1151. In some embodiments, tube 1154 is heat shrunk onto both lens assembly 1151 and plug 1153, such that tube 1154 is arranged in a profile similar to that which is illustrated in FIG. 10B, such that space 1152 comprises a variable outer diameter, and at least a portion of that diameter is less than the outer diameter of lens assembly 1151. In some embodiments, tube 1154 does not come into contact with the beveled distal end of lens assembly 1151, for example, such that only the fluid within space 1152 contacts the distal end of lens assembly 1151, ensuring a total internal reflection of the distal end of lens assembly 1151 (e.g. due to the glass-air interface at the distal end of lens assembly 1151). In some embodiments, the reduced diameter of plug 1153 allows imaging probe 100 to achieve a tighter (i.e. smaller) bend radius than would be achievable if plug 1153 comprised the same or a larger outer diameter as lens assembly 1151.

FIG. 10C shows a perspective view of a portion of shaft 120 with optical core 110 removed for illustrative clarity. Shaft 120 can comprise at least a first (proximal) portion, tube 121, which is fixedly attached via joint 125 to a second (distal) portion, window 130. Tube 121 can comprise an elongate hollow member, such as a hypotube (e.g. a metal tube that can include one or more engineered features along its length). In some embodiments, tube 121 comprises a hypotube including a spiral cut along at least a portion of its length. In some embodiments, tube 121 comprises a spiral cut with a strain relief on the proximal end of the spiral cut that is configured to reduce the likelihood of plastic deformation at the end of the spiral cut. The strain relief can comprise an end with a radius larger than the width of the remaining portion of the spiral cut. This strain relief reduces the stress concentration on tube 121 (e.g. when that region of tube 121 is bent). In some embodiments, tube 121 comprises nickel-titanium alloy (e.g. super elastic nickel-titanium alloy). In some embodiments, tube 121 comprises a plastic material, such as polyimide and/or PEEK. In some embodiments, window 130 comprises a transparent elongate hollow member, such as is described hereabove in reference to FIG. 1. Joint 125 can comprise at least the distal portion of tube 121 and at least the proximal portion of window 130. The distal portion of tube 121 can comprise a tapered portion, taper 1211. Taper 1211 can be configured such that the outer diameter of tube 121 decreases from the proximal end of taper 1211 to the distal end of taper 1211. Tube 121 can comprise a lumen 1215 therethrough (not shown).

An elongate segment, tube 123, is partially inserted into lumen 1215 of tube 121. Tube 123 can comprise an outer diameter approximately equal to the diameter of lumen 1215. In some embodiments, tube 123 comprises an outer diameter slightly greater than the diameter of lumen 1215 (e.g. such that tube 123 can be press fit into lumen 1215). Alternatively, tube 123 can comprise a diameter equal to or slightly smaller than the diameter of lumen 1215 (e.g. such as to provide space for an adhesive between tube 123 and tube 121). Tube 123 can comprise a lumen 1235 therethrough. In some embodiments, lumen 1235 comprises a diameter greater than the outer diameter of optical core 110 (e.g. such that optical core 110 can be slidingly positioned therethrough). Additionally or alternatively, lumen 1235 can comprise a diameter (e.g. tube 123 comprises an inner diameter) that is less than the outer diameter of FPE 1500, such that FPE 1500 (not shown in FIG. 10C) cannot translate proximally beyond tube 123 (e.g. translation of FPE 1500 proximally causes FPE 1500 to contact the end of tube 123, preventing FPE 1500 from entering lumen 1235). In some embodiments, the proximal portion of window 130 slidingly receives the distal portion of tube 123. The proximal portion of window 130 can further slidingly receive at least a distal portion of taper 1211 of tube 121. Window 130 can be fixedly attached to tubes 121 and/or 123 via compression and/or an adhesive. In some embodiments, the proximal end of window 130 is reduced (e.g. shrunk) onto tubes 121 and/or 123 to provide a compression joint. In some embodiments, window 130 comprises a material configured to contract when under tension (e.g. when window 130 is pulled, intentionally or unintentionally away from tube 121), such that window 130 compresses onto tubes 121 and/or 123, increasing the retention force of joint 125 when tension is applied. In these embodiments, tubes 121 and/or 123 can comprise a lower modulus than window 130, such that window 130 compresses more under equal tension than one or more of tubes 121 and/or 123. In some embodiments, joint 125 comprises a construction and arrangement similar to joint 125 as described in applicants co-pending International PCT Patent Application Serial Number PCT/US2020/030616, titled “Imaging Probe with Fluid Pressurization Element”, filed Apr. 30, 2020, the content of which is incorporated herein by reference in its entirety for all purposes.

In some embodiments, marker 131 is positioned between tube 123 and window 130. In some embodiments, marker 131 is positioned at least 0.5 mm from taper 1211, such as to prevent a stiff section of shaft 120 from being created within joint 125. Marker 131 can comprise a radiopaque marker configured to be imaged using fluoroscopy or other X-ray based imaging. Marker 131 can comprise an outer diameter of no more than 0.25 mm. In some embodiments, marker 131 is positioned a distance D_{P} from the distal end of spring tip 119 (as shown in FIG. 10B). In some embodiments, distance D_{P} comprises at least the maximum pullback distance enabled by system 10. In some embodiments, D_{P} comprises a length of approximately 49 mm. This placement can be selected such that marker 131 enables the operator of system 10 to know, prior to the performance of the pullback procedure, the location where the distal end of spring tip 119 will be after the pullback procedure has been performed (e.g. to prevent the distal end of spring tip 119 to be positioned after the pullback procedure at an undesirably proximal location or other undesirable location).

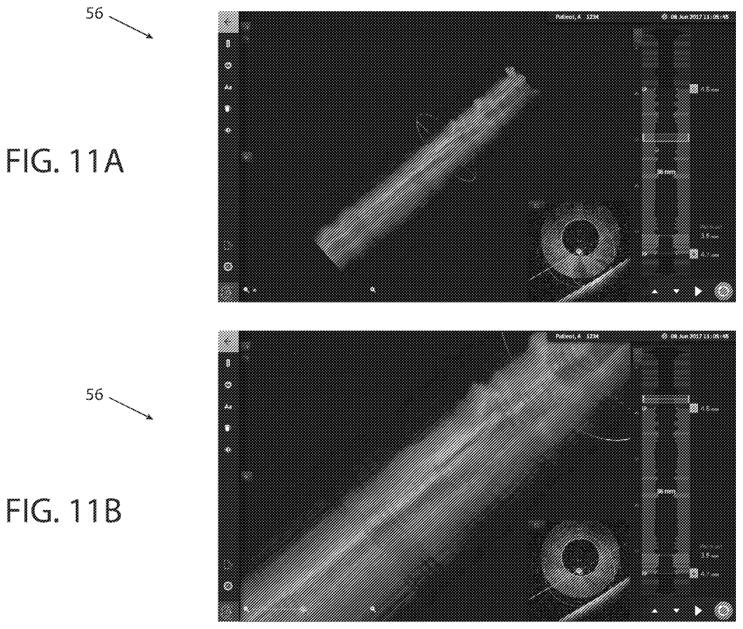

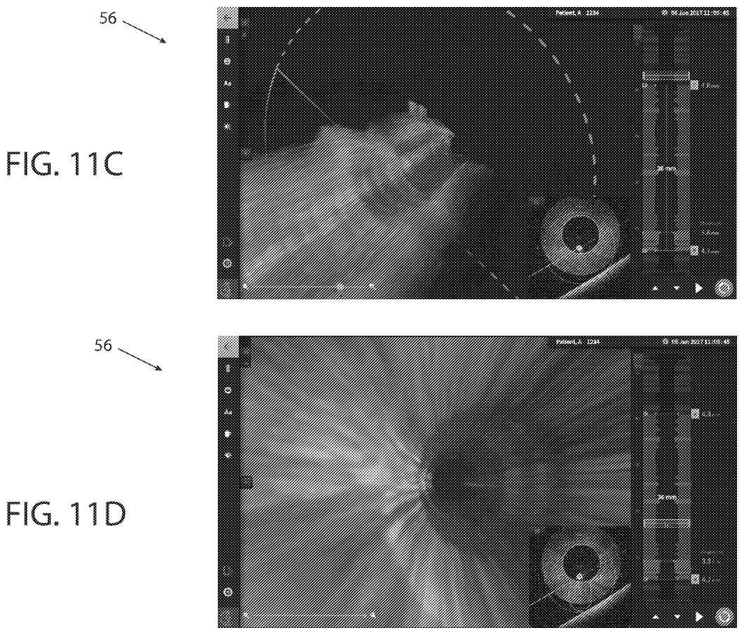

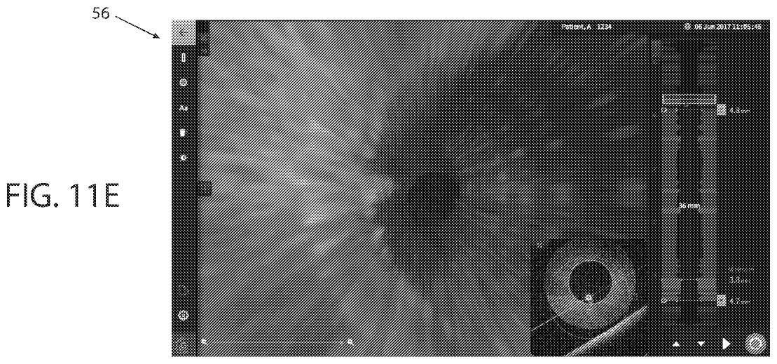

Referring now to FIGS. 11A-11E, five displays of lumen imaging data are illustrated, consistent with the present inventive concepts. System 10 can display images comprising a graphical representation of OCT image data and/or other image data collected by and/or input into system 10 (e.g. angiographic image data, MRI image data, or the like). The images shown in FIGS. 11A-11E comprise images produced by the imaging probes and other components of the imaging systems of the present inventive concepts. The images shown in FIGS. 11A-E can be displayed to an operator (e.g. on display 56 of system 10 described herein). In FIG. 11A, an oblique representation of lumen image data is displayed (e.g. a sectional view in which the viewer is looking at the vessel from an oblique angle outside of the vessel). In this view, half of the imaged vessel is cut away such that the inside of the vessel can be displayed from this oblique angle. In FIG. 11B, the image displayed in FIG. 11A is displayed zoomed in, for example with approximately 50% magnification. In FIG. 11C, a transitional display is illustrated. The image is shown transitioning from an oblique sectional representation of the image data to a “fly-through” representation, meaning the image is displayed as if the viewer's perspective is looking along the vessel from the inside (e.g. from the center of the vessel). In FIG. 11D, the image data is being displayed in a fly through representation from a first axial location of the imaged lumen, and in FIG. 11E, the image data is being displayed in a fly-through representation from a second axial location of the imaged lumen. In some embodiments, the axial location from which the vessel is displayed in the fly-through representation is linked with the position shown in other views displayed simultaneously on display 56, such as when B-mode or L-mode view (e.g. as known in the art) is simultaneously displayed. In some embodiments, system 10 provides a zoom functionality, where the display automatically transitions (e.g. as illustrated in FIG. 11C) from a cross sectional representation to a fly-through representation once the operator zooms in past a threshold, such as past a 70% magnification threshold. Alternatively or additionally, the operator can selectively zoom to transition between the two display modes (for example by selecting a keyboard modifier key, such as the shift key). In some embodiments, the operator can “virtually travel” through the lumen by advancing the perspective location of the fly-through image. In some embodiments, the same operator input (e.g. provided via a user input with a single degree of freedom, such as a mouse wheel) controls both the cross sectional zoom and the fly-through advancement, for example, with a keyboard modifier. In some embodiments, the zoom functionality is executed in the oblique representation by system 10 by “dollying” the camera (e.g. the point from which the viewer is viewing the vessel) towards and/or away from the vessel, such as towards and/or away from the center of a selected axial location of the displayed vessel. As the oblique representation zoom is increased beyond a threshold, the camera begins to transition toward the fly-through orientation (e.g. the oblique angle decreases) until the camera (e.g. the viewer's perspective) is positioned along the centerline of the lumen facing along the longitudinal axis of the vessel. As the oblique angle decreases, the cut away portion of the vessel is also displayed with increasing opacity, for example, where the display reaches full opacity once the camera reaches the centerline of the vessel. In some embodiments, zooming out causes the reverse to occur.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. An imaging system for a patient comprising:
an imaging probe, comprising:
an elongate shaft comprising a proximal end, a distal portion, and a lumen extending between the proximal end and the distal portion;
a rotatable optical core comprising a proximal end and a distal end, wherein at least a portion of the rotatable optical core is positioned within the lumen of the elongate shaft; and
an optical assembly positioned proximate the distal end of the rotatable optical core, the optical assembly configured to direct light to tissue to be imaged, and to collect reflected light from the tissue to be imaged; and
an imaging assembly constructed and arranged to optically couple to the imaging probe, the imaging assembly configured to emit light into the imaging probe and receive the reflected light collected by the optical assembly.

2-21. (canceled)
